(19) United States
(12) Patent Application Publication
(10) Pub. No.: US 2024/0024110 A1
(43) Pub. Date: Jan. 25, 2024

(54) DELIVERY SYSTEMS FOR IMPLANTS

(71) Applicant: EDWARDS LIFESCIENCES CORPORATION, Irvine, CA (US)

(72) Inventors: Corey Maurice Marshall, Newport Beach, CA (US); Yuanlong Du, Irvine, CA (US); Tri D. Tran, Fountain Valley, CA (US); Ronaldo C. Cayabyab, Mission Viejo, CA (US)

(21) Appl. No.: 18/477,405

(22) Filed: Sep. 28, 2023

Related U.S. Application Data
(63) Continuation of application No. PCT/US2022/024563, filed on Apr. 13, 2022.
(60) Provisional application No. 63/174,712, filed on Apr. 14, 2021.

(51) Int. Cl. A61F 2/24; A61M 25/01

(52) U.S. Cl. CPC A61F 2/2466; A61M 25/0147; A61M 2025/0161

(57) ABSTRACT

A delivery catheter is in various examples configured to deliver an anchoring device to a native valve annulus of a patient's heart, where the anchoring device can better secure a prosthesis at the native annulus. The delivery catheter in examples may be configured to deflect in a ventricular direction during deployment of an anchoring device. Examples of docking coil sleeves and docking coils are disclosed herein.

### CROSS-REFERENCE TO RELATED APPLICATIONS

This application is a continuation of PCT patent application no. PCT/US2022/024563 filed on Apr. 13, 2022, which application claims the benefit of U.S. Provisional Application No. 63/174,712 filed on Apr. 14, 2021, the entire contents of each of these applications being incorporated herein by reference.

### BACKGROUND

### Field

The present disclosure generally concerns deployment tools for delivering anchoring devices, such as prosthetic docking devices that support prostheses and methods of using the same. For example, the disclosure relates to replacement of heart valves that have malformations and/or dysfunctions, where a delivery catheter is utilized to deploy anchoring devices that support a prosthetic heart valve at an implantation site, and methods of using the delivery catheter to implant such anchoring devices and/or prosthetic heart valves.

### Background

Referring generally to FIGS. 1A-1B, the native mitral valve 50 controls the flow of blood from the left atrium 51 to the left ventricle 52 of the human heart and, similarly, the tricuspid valve 59 controls the flow of blood between the right atrium 56 and the right ventricle 61. The mitral valve has a different anatomy than other native heart valves. The mitral valve includes an annulus made up of native valve tissue surrounding the mitral valve orifice, and a pair of cusps or leaflets extending downward from the annulus into the left ventricle. The mitral valve annulus can form a “D” shaped, oval shaped, or otherwise non-circular cross-sectional shape having major and minor axes. An anterior leaflet can be larger than a posterior leaflet of the valve, forming a generally “C” shaped boundary between the abutting free edges of the leaflets when they are closed together.

When operating properly, the anterior leaflet 54 and the posterior leaflet 53 of the mitral valve function together as a one-way valve to allow blood to flow from the left atrium 51 to the left ventricle 52. After the left atrium receives oxygenated blood from the pulmonary veins, the muscles of the left atrium contract and the left ventricle relaxes (also referred to as “ventricular diastole” or “diastole”), and the oxygenated blood that is collected in the left atrium flows into the left ventricle. Then, the muscles of the left atrium relax and the muscles of the left ventricle contract (also referred to as “ventricular systole” or “systole”), to move the oxygenated blood out of the left ventricle 52 and through the aortic valve 63 and the aorta 58 to the rest of the body. The increased blood pressure in the left ventricle during ventricular systole urges the two leaflets of the mitral valve together, thereby closing the one-way mitral valve so that blood cannot flow back into the left atrium. To prevent or inhibit the two leaflets from prolapsing under the pressure and folding back through the mitral annulus toward the left atrium during ventricular systole, a plurality of fibrous cords 62 called chordae tendineae tether the leaflets to papillary muscles in the left ventricle. The chordae tendineae 62 are schematically illustrated in both the heart cross-section of FIG. 1A and the top view of the mitral valve in FIG. 1B.

Problems with the proper functioning of the mitral valve are a type of valvular heart disease. Valvular heart disease can affect the other heart valves as well, including the tricuspid valve. A common form of valvular heart disease is valve leak, also known as regurgitation, which can occur in various heart valves, including both the mitral and tricuspid valves. Mitral regurgitation occurs when the native mitral valve fails to close properly and blood flows back into the left atrium from the left ventricle during ventricular systole. Mitral regurgitation can have different causes, such as leaflet prolapse, dysfunctional papillary muscles, problems with chordae tendineae, and/or stretching of the mitral valve annulus resulting from dilation of the left ventricle. In addition to mitral regurgitation, mitral narrowing or stenosis is another example of valvular heart disease. In tricuspid regurgitation, the tricuspid valve fails to close properly and blood flows back into the right atrium from the right ventricle.

Like the mitral and tricuspid valves, the aortic valve is likewise susceptible to complications, such as aortic valve stenosis or aortic valve insufficiency. One method for treating aortic heart disease includes the use of a prosthetic valve implanted within the native aortic valve. These prosthetic valves can be implanted using a variety of techniques, including various transcatheter techniques. A transcatheter heart valve (THV) can be mounted in a crimped state on the end portion of a flexible and/or steerable catheter, advanced to the implantation site in the heart via a blood vessel connected to the heart, and then expanded to its functional size, for example, by inflating a balloon on which the THV is mounted. Alternatively, a self-expanding THV can be retained in a radially compressed state within a sheath of a delivery catheter, where the THV can be deployed from the sheath, which allows the THV to expand to its functional state. Such delivery catheters and techniques of implantation are generally more developed for implantation or use at the aortic valve, but do not address the unique anatomy and challenges of other valves.

### SUMMARY

This summary is meant to provide some examples and is not intended to be limiting of the scope of the disclosure in any way. For example, any feature included in an example of this summary is not required by the claims, unless the claims explicitly recite the features. Also, the features described can be combined in a variety of ways. Various features and steps as described elsewhere in this disclosure may be included in the examples summarized here.

Tools and methods are provided for mitral and tricuspid valve replacements, including for adapting different types of implants such as valve or valves (e.g., those designed for aortic valve replacement or other locations) for use at the mitral and tricuspid valve locations. One way of adapting these other prosthetic valves at the mitral position or tricuspid position is to deploy the prosthetic valves into an implant such as an anchor or other docking device/station that will form a more appropriately shaped implant site at the native valve annulus. The anchor or other docking device/stations herein allow a prosthetic valve to be implanted more securely, while also reducing or eliminating leakage around the valve after implantation.

One type of implant in the form of an anchor or anchoring device that can be used herein is a docking coil including a coil or helically shaped anchor that provides for a circular or cylindrical docking site for cylindrically shaped prosthetic valves. One type of anchor or anchoring device that can be used herein includes a coiled region and/or helically shaped region that provides for a circular or cylindrical docking site for cylindrically shaped prosthetic valves. In this manner, optionally an existing valve implant developed for the aortic position, perhaps with some modification, can be implanted at another valve position such as the mitral position together with such an anchor or anchoring device. Such anchors or anchoring devices can be used at the heart's other native valves, such as the tricuspid valve, to more securely anchor prosthetic valves at those sites as well.

Described herein are examples of deployment tools to assist in delivering implants in the form of prosthetic devices at one of the native mitral, aortic, tricuspid, or pulmonary valve regions of a human heart, as well as methods for using the same. The disclosed deployment tools can be used to deploy implants in the form of anchoring devices (e.g., prostheses docking devices, prosthetic valve docking devices, etc.), such as helical anchoring devices or anchoring devices having a plurality of turns or coils, at the implantation site to provide a foundational support structure into which a prosthetic heart valve can be implanted. A delivery catheter may comprise a steerable delivery catheter in examples.

In examples, a system is disclosed. The system may comprise a system for delivering an implant to a portion of a patient's body. The system may comprise a delivery catheter including an elongate shaft having an interior lumen for the implant to pass through and a distal end portion including a first flexible portion and a second flexible portion that is positioned distal of the first flexible portion.

The first flexible portion may include a first tether and a first linear spine that is positioned opposed circumferentially to the first tether, and the first flexible portion is configured to deflect in a plane upon a longitudinal force being applied to the first tether.

The second flexible portion may include a second tether and a second linear spine that is positioned non-orthogonal and non-parallel relative to the first linear spine, and the second flexible portion is configured to deflect in a direction that is non-orthogonal and non-parallel with the plane upon a longitudinal force being applied to the second tether.

In examples, a system is disclosed. The system may comprise a system for delivering an implant to a portion of a patient's body. The system may comprise a delivery catheter including an elongate shaft having an interior lumen for the implant to pass through and a distal end portion including a flexible portion with a tether and a linear spine that is positioned at an obtuse angle circumferentially from the tether. The flexible portion may be configured to deflect to form a curve upon a longitudinal force being applied to the tether.

In examples, a system is disclosed. The system may comprise a system for delivering an implant to a portion of a patient's body. The system may comprise a delivery catheter including an elongate shaft having an interior lumen for the implant to pass through and a distal end portion including a first flexible portion and a second flexible portion that is positioned distal of the first flexible portion.

The first flexible portion may include a first tether and a first linear spine that is positioned opposed circumferentially to the first tether, and the first flexible portion is configured to deflect in a first plane upon a longitudinal force being applied to the first tether.

The second flexible portion may include a second tether positioned orthogonal relative to the first tether and a second linear spine positioned opposed circumferentially to the second tether, and a third tether positioned opposed circumferentially relative to the first tether, and the second flexible portion is configured to deflect in a second plane that is orthogonal to the first plane upon a longitudinal force being applied to the second tether and the second flexible portion is configured to deflect in the first plane upon a longitudinal force being applied to the third tether.

In examples, a system is disclosed. The system may include a docking coil configured to dock with an implant within a portion of a patient's body. The system may include a docking coil sleeve having an interior lumen configured for the docking coil to slide within and including a tether extending along at least a portion of the docking coil sleeve and configured to deflect the docking coil sleeve.

In examples, a system is disclosed. The system may include a docking coil configured to dock with an implant within a portion of a patient's body and including a leading portion extending to a leading tip and having an orientation.

The system may include a docking coil sleeve having an interior lumen configured for the docking coil to slide within and including a leading portion extending to a leading tip and having an orientation that is different than the orientation of the leading portion of the docking coil, the leading tip of the docking coil sleeve configured to slide relative to the leading tip of the docking coil to deflect the leading tip of the docking coil or the leading tip of the docking coil sleeve radially inward or outward.

In examples, a method is disclosed. The method may include advancing a delivery catheter to a position within a patient's body. The delivery catheter may include an elongate shaft having an interior lumen for an implant to pass through and a distal end portion including a first flexible portion and a second flexible portion that is positioned distal of the first flexible portion.

The first flexible portion may include a first tether and a first linear spine that is positioned opposed circumferentially to the first tether, and the first flexible portion is configured to deflect in a plane upon a longitudinal force being applied to the first tether.

The second flexible portion including a second tether and a second linear spine that is positioned non-orthogonal and non-parallel relative to the first linear spine, and the second flexible portion is configured to deflect in a direction that is non-orthogonal and non-parallel with the plane upon a longitudinal force being applied to the second tether.

The method may include deploying the implant from the interior lumen to an implantation site within the patient's body.

In examples, a method is disclosed. The method may include advancing a delivery catheter to a position within a patient's body. The delivery catheter may include an elongate shaft having an interior lumen for an implant to pass through and a distal end portion including a first flexible portion and a second flexible portion that is positioned distal of the first flexible portion.

The first flexible portion may include a first tether and a first linear spine that is positioned opposed circumferentially to the first tether, and the first flexible portion is configured to deflect in a first plane upon a longitudinal force being applied to the first tether.

The second flexible portion may include a second tether positioned orthogonal relative to the first tether and a second linear spine positioned opposed circumferentially to the second tether, and a third tether positioned opposed circumferentially relative to the first tether, and the second flexible portion is configured to deflect in a second plane that is orthogonal to the first plane upon a longitudinal force being applied to the second tether and the second flexible portion is configured to deflect in the first plane upon a longitudinal force being applied to the third tether.

The method may include deploying the implant from the interior lumen to an implantation site within the patient's body.

In examples, a method is disclosed. The method may include deploying a docking coil from a docking coil sleeve to an implantation site within a patient's body, the docking coil being configured to dock with an implant within the patient's body, and the docking coil sleeve having an interior lumen configured for the docking coil to slide within and including a tether extending along at least a portion of the docking coil sleeve and configured to deflect the docking coil sleeve.

In examples, a method is disclosed. The method may include deploying a docking coil from a docking coil sleeve to an implantation site within a patient's body, the docking coil configured to dock with an implant within a portion of a patient's body and including a leading portion extending to a leading tip and having an orientation.

The docking coil sleeve may have an interior lumen configured for the docking coil to slide within and including a leading portion extending to a leading tip and having an orientation that is different than the orientation of the leading portion of the docking coil, the leading tip of the docking coil sleeve configured to slide relative to the leading tip of the docking coil to deflect the leading tip of the docking coil or the leading tip of the docking coil sleeve radially inward or outward.

### BRIEF DESCRIPTION OF THE DRAWINGS

The foregoing and other objects, features, and advantages of the disclosure will become more apparent from the following detailed description using the accompanying figures. In the drawings:

FIG. 1A shows a schematic cross-sectional view of a human heart.

FIG. 1B shows a schematic top view of the mitral valve annulus of a heart.

FIG. 2 shows a partial perspective view of an exemplary delivery catheter for implanting an implant in the form of an anchoring device at a native valve of a heart, using a transseptal technique.

FIG. 3 shows a cross-sectional view of an anchoring device and an exemplary prosthetic heart valve implanted at the native valve of the heart.

FIG. 4 shows a side view of a delivery catheter.

FIG. 5A shows a cross sectional view of a portion of a delivery catheter.

FIG. 5B shows a cross sectional view along line 5B-5B of the delivery catheter.

FIG. 5C shows a cross sectional view along line 5C-5C of the delivery catheter.

FIG. 6 shows a side cross sectional view of a spine.

FIG. 7A illustrates a top view of a distal end portion of a catheter.

FIG. 7B illustrates an end view of the catheter shown in FIG. 7A.

FIG. 7C illustrates a top view of a distal end portion of a catheter deflected from the position shown in FIG. 7A.

FIG. 7D illustrates an end view of the catheter in the position shown in FIG. 7C.

FIG. 8A illustrates an end view of a catheter.

FIG. 8B illustrates a top view of the catheter shown in FIG. 8A.

FIG. 8C illustrates a top view of the catheter shown in FIG. 8B with a distal end portion of the catheter deflected.

FIG. 8D illustrates an end view of the catheter shown in FIG. 8C.

FIG. 8E illustrates a side view of the catheter shown in FIG. 8D.

FIG. 9A shows a cross sectional view of a portion of a delivery catheter.

FIG. 9B shows a cross sectional view along line 9B-9B of the delivery catheter.

FIG. 9C shows a cross sectional view along line 9C-9C of the delivery catheter.

FIG. 10A shows a cross sectional view of a portion of a delivery catheter.

FIG. 10B shows a cross sectional view along line 10B-10B of the delivery catheter.

FIG. 10C shows a cross sectional view along line 10C-10C of the delivery catheter.

FIG. 11A is a side cutout view of a portion of a patient's heart that illustrates an exemplary delivery catheter entering the left atrium through the fossa ovalis in an exemplary method.

FIG. 11B illustrates the delivery catheter of FIG. 11A entering the left atrium of the patient's heart in the position shown in FIG. 11A, in which the delivery device is shown from a view taken along the lines 11B-11B in FIG. 11A.

FIG. 12A illustrates the delivery device of FIG. 11A in a position.

FIG. 12B illustrates the delivery device of FIG. 11A in the position shown in FIG. 12A, in which the delivery device is shown from a view taken along the lines 12B-12B in FIG. 12A.

FIG. 13A illustrates a side perspective view of a docking coil.

FIG. 13B illustrates a top view of the docking coil shown in FIG. 13A.

FIG. 14A illustrates a side view of a docking coil sheath.

FIG. 14B illustrates a side view of the docking coil sheath shown in FIG. 14A with a portion shown in cross section.

FIG. 14C illustrates a cross section of the docking coil sheath shown in FIG. 14B along line 14C-14C.

FIG. 15A illustrates a side view of a docking coil sheath with a portion shown in cross section.

FIG. 15B illustrates a cross section of the docking coil sheath shown in FIG. 15A along line 15B-15B.

FIG. 16A illustrates a side view of a docking coil sheath with a portion shown in cross section.

FIG. 16B illustrates a cross section of the docking coil sheath shown in FIG. 16A along line 16B-16B.

FIG. 17A illustrates a cross sectional view of a portion of a docking coil sheath.

FIG. 17B illustrates a top schematic view of a docking coil sheath extending around a mitral valve.

FIG. 17C illustrates a side cross sectional view of a mitral valve with a docking coil and docking coil sheath extending around the mitral valve.

FIG. 18A illustrates a side perspective view of a docking coil.

FIG. 18B illustrates a top view of the docking coil shown in FIG. 18A.

FIG. 19A illustrates a side view of a leading portion of a docking coil and a leading portion of a docking coil sheath.

FIG. 19B illustrates a cross sectional view of a docking coil positioned within a docking coil sheath.

FIG. 19C illustrates a cross sectional view of a docking coil positioned within a docking coil sheath and deflecting the docking coil sheath.

FIG. 20 illustrates a cross sectional view of a docking coil sheath.

FIG. 21A illustrates a cross sectional view of a docking coil sheath.

FIG. 21B illustrates a cross sectional view of a docking coil extending within a docking coil sheath.

FIG. 22A illustrates a schematic view of a docking coil extending within a docking coil sheath.

FIG. 22B illustrates a schematic view of a docking coil deflecting a docking coil sheath.

FIG. 23A illustrates a side view of a docking coil sheath.

FIG. 23B illustrates a cross sectional view of a docking coil within a docking coil sheath.

FIG. 23C illustrates a cross sectional view of a docking coil within a docking coil sheath.

FIG. 24A is a side cutout view of the left side of a patient's heart that illustrates an anchoring device being delivered around the chordae tendineae and leaflets in the left ventricle of the patient's heart.

FIG. 24B illustrates the anchoring device of FIG. 24A further wrapping around the chordae tendineae and leaflets in the left ventricle of the patient's heart as it is being delivered by the delivery catheter of FIG. 24A.

FIG. 24C illustrates the anchoring device of FIG. 24A further wrapping around the chordae tendineae and leaflets in the left ventricle of the patient's heart as it is being delivered by the delivery catheter of FIG. 24A.

FIG. 24D is a view looking down into the patient's left atrium, illustrating a delivery catheter, after an anchoring device is wrapped around the chordae tendineae and leaflets in the left ventricle of the patient's heart.

FIG. 24E illustrates a delivery catheter in the left atrium of the patient's heart, in which the delivery catheter is retracting to deliver a portion of the anchoring device in the left atrium of the patient's heart.

FIG. 24F illustrates a delivery catheter in the left atrium of the patient's heart, in which the delivery catheter is retracting to deliver a further portion of the anchoring device in the left atrium of the patient's heart.

FIG. 24G illustrates a delivery catheter in the left atrium of the patient's heart, in which the anchoring device is exposed and shown connected tightly to a pusher in the left atrium of the patient's heart.

FIG. 24H illustrates the delivery catheter in the left atrium of the patient's heart, in which the anchoring device is fully removed from the delivery device and is loosely and removably attached to the pusher by a suture.

FIG. 24I is a cutout view of the patient's heart that illustrates an exemplary example of a prosthetic heart valve being delivered by an exemplary example of a heart valve delivery device to the mitral valve of the patient.

FIG. 24J illustrates the heart valve of FIG. 24I being further delivered to the mitral valve of the patient by the heart valve delivery device.

FIG. 24K illustrates the heart valve of FIG. 24I being opened by inflation of a balloon to expand and attach the heart valve to the mitral valve of the patient.

FIG. 24L illustrates the heart valve of FIG. 24I attached to the mitral valve of the patient's heart and secured by an anchoring device.

FIG. 24M is an upward view of the mitral valve from the left ventricle that illustrates the prosthetic heart valve of FIG. 24I attached to the mitral valve of the patient's heart from a view taken along the lines 24M-24M in FIG. 24L.

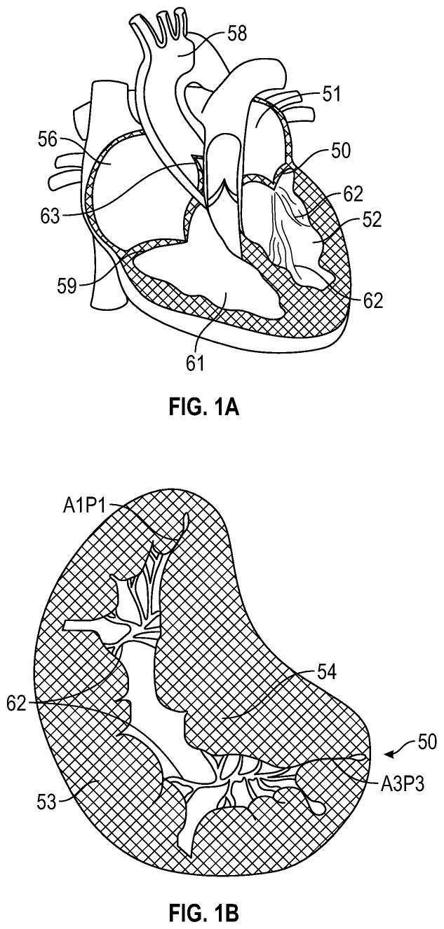

### DETAILED DESCRIPTION

The following description and accompanying figures, which describe and show certain examples, are made to demonstrate, in a non-limiting manner, several possible configurations of systems, devices, apparatuses, components, methods, etc. that may be used for various aspects and features of the present disclosure. As one example, various systems, devices/apparatuses, components, methods, etc. are described herein that may relate to mitral valve procedures. However, specific examples provided are not intended to be limiting, e.g., the systems, devices/apparatuses, components, methods, etc. can be adapted for use in other valves beyond the mitral valve (e.g., in the tricuspid valve).

Described herein are examples of deployment tools that are intended to facilitate implantation of implants in the form of prosthetic devices (e.g., prosthetic valves) at one of the native mitral, aortic, tricuspid, or pulmonary valve regions of a human heart, as well as methods for using the same. The prosthetic devices or valves can be expandable transcatheter heart valves (“THVs”) (e.g., balloon expandable, self-expandable, and/or mechanically expandable THVs). The deployment tools can be used to deploy anchoring devices (sometimes referred to as docking devices, docking stations, or similar terms) that provide a more stable docking site to secure the prosthetic device or valve (e.g., THVs) at the native valve region. The anchoring devices may comprise docking coils in examples. These deployment tools can be used to more accurately place such anchoring devices (e.g., prostheses anchoring devices, prosthetic valve anchoring device, etc.), so that the anchoring devices and any prostheses (e.g., prosthetic devices or prosthetic heart valves) anchored thereto function properly after implantation.

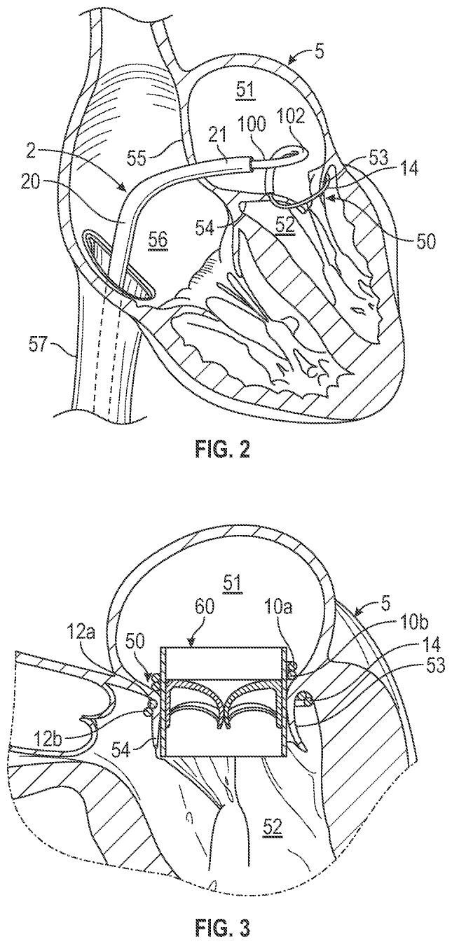

FIG. 2 shows a delivery device 2 for installing an implant in the form of an anchoring device 14 at a native mitral valve annulus 50 using a transseptal technique. The same or a similar delivery device 2 could be used to deliver an anchoring device 14 at the tricuspid valve without having to leave the right atrium to cross the septum into the left atrium. The delivery device 2 includes a sheath catheter including an outer sheath or guide sheath 20. The delivery device 2 includes a delivery catheter 100. The guide sheath 20 has a shaft in the shape of an elongated hollow tube through which the delivery catheter 100, as well as various other components (e.g., implants such as the anchoring device and a prosthetic heart valve, etc.), can pass, thus allowing the components to be introduced into the patient's heart 5. The guide sheath 20 can be steerable so that the guide sheath 20 can be bent at various angles needed for the guide sheath 20 to pass through the heart 5 and enter the left atrium 51. The sheath 20 may comprise a steerable guide sheath including a lumen for a delivery catheter to pass through. The steerable guide sheath may be configured to deflect a portion of an elongate shaft of the delivery catheter 100 when the elongate shaft is positioned within the lumen of the steerable guide sheath. While in the guide sheath 20, the delivery catheter 100 has a relatively straight or straightened shape (compared to a curved shape discussed in greater detail below), e.g., the delivery catheter 100 is held in guide sheath 20 in a configuration or shape that corresponds to the configuration or shape of the guide sheath 20.

Like the guide sheath 20, the delivery catheter 100 has an elongate shaft having the shape of an elongated hollow tube. However, the delivery catheter 100 has a smaller diameter than the sheath 20 so that it can slide axially within the sheath 20. Meanwhile, the delivery catheter 100 is large enough to house and deploy an implant such as an anchoring device, such as a docking coil.

The elongate shaft of the delivery catheter 100 may have a distal end portion 102. The distal end portion 102 may bend into a configuration that allows for more accurate placement of an anchoring device, such as a docking coil, and may allow the distal end portion 102 to be held at such configuration. For example, the distal end portion 102 may bend into a curved shape to assist in extrusion or pushing out of an anchoring device on a ventricular side of the mitral valve 50, so that the lower coils (e.g., functional coils and/or encircling coils) of the anchoring device 14 can be properly installed below the annulus of the native valve. The distal end portion 102 can also be bent into a curved shape so that the upper coil(s) (e.g., a stabilization coil/turn or upper coils) of the anchoring device can be accurately deployed on the atrial side of the annulus of the native valve. For example, the distal end portion 102 can have a curved shape for installing upper coils and a curved shape for installing lower coils. In other examples, the distal end portion 102 may have one configuration for installing lower coils and another configuration for installing the upper coils.

FIG. 3, for example, illustrates an anchoring device in the form of a docking coil positioned around the mitral valve, with a prosthetic valve, for example, a prosthetic transcatheter heart valve (THV) 60 docked with the anchoring device. The anchoring device 14 is implanted so that one or more upper coils/turns (e.g., the upper coils 10a, 10b) are above, i.e., on the atrial side, of the annulus of the native valve (e.g., mitral valve 50 or a tricuspid valve) and the lower coils 12a, 12b are below, i.e., on the ventricular side, of the annulus of the native valve. In this configuration, the mitral leaflets 53, 54 can be captured between the upper coils 10a, 10b and the lower coils 12a, 12b. When implanted, the various anchoring devices herein can provide a solid support structure to secure the prosthetic valve in place and avoid migration due to the operation of the heart.

Referring to FIG. 2, in a method of deployment, when using a transseptal delivery method to access the mitral valve, the guide sheath 20 can be inserted through a femoral vein, through the inferior vena cava 57 and into the right atrium 56. Alternatively, the guide sheath 20 can be inserted through a jugular vein or subclavian vein or other upper vasculature location and passed through the superior vena cava and into the right atrium. The interatrial septum 55 is then punctured (e.g., at the fossa ovalis) and the sheath 20 is passed into the left atrium 51, as can be seen in FIG. 2. (In tricuspid valve procedures, it is unnecessary to puncture or cross the septum 55.)

In mitral valve procedures, with the sheath 20 in position in the left atrium 51, the delivery catheter 100 is advanced from the distal end 21 of the sheath 20, such that the distal end portion 102 of the delivery catheter 100 is also in the left atrium 51. In this position, the distal end portion 102 of the delivery catheter 100 can be curved to allow for an anchoring device 14 to be installed at the annulus of the mitral valve 50. The anchoring device 14 can then be advanced through the delivery catheter 100 and installed at the mitral valve 50. The anchoring device 14 can be attached to a pusher that advances or pushes the anchoring device 14 through the delivery catheter 100 for implantation. The pusher can be a wire or tube with sufficient strength and physical characteristics to push the anchoring device 14 through the delivery catheter 100. In some examples, the pusher can be made of or include a spring or coil, a tube extrusion, a braided tube, or a laser cut hypotube, among other structures. In some examples, the pusher can have a coating over and/or inside it, e.g., it can have an interior lumen lined by PTFE to allow a line (e.g., a suture) to be atraumatically actuated through the lined lumen. As noted above, in some examples, after the pusher has pushed and properly positioned the ventricular coils of the anchoring device 14 in the left ventricle, the distal end portion 102 can be moved to release the atrial coils of the anchoring device 14 into the left atrium, while maintaining or holding a position of the ventricular coils of the anchoring device 14 within the left ventricle.

Once the anchoring device 14 is installed, the delivery catheter 100 can be removed by straightening or reducing the curvature of the distal end portion 102 to allow the delivery catheter 100 to pass back through the guide sheath 20. With the delivery catheter 100 removed, a prosthetic valve, for example, a prosthetic transcatheter heart valve (THV) 60 can then be passed, for example, through the guide sheath 20 and secured within the anchoring device 14, as shown for example in FIG. 3. When the THV 60 is secured within the anchoring device 14, the guide sheath 20 along with any other delivery apparatuses for the THV 60 can then be removed from the patient's body and the openings in the patient's septum 55 and right femoral vein can be closed. In other examples, after the anchoring device 14 has been implanted, a different sheath or different delivery device altogether can be separately used to deliver the THV 60. For example, a guide wire can be introduced through guide sheath 20, or the guide sheath 20 can be removed and the guide wire can be advanced via the same access point, through the native mitral valve, and into the left ventricle, using a separate delivery catheter. Meanwhile, even though the anchoring device is implanted transseptally in this example, it is not limited to transseptal implantation, and delivery of the THV 60 is not limited to transseptal delivery (or more generally via the same access point as delivery of the anchoring device). In still other examples, after transseptal delivery of the anchoring device 14, any of various other access points can thereafter be used to implant the THV 60, for example, trans-apically, trans-atrially, or via the femoral artery.

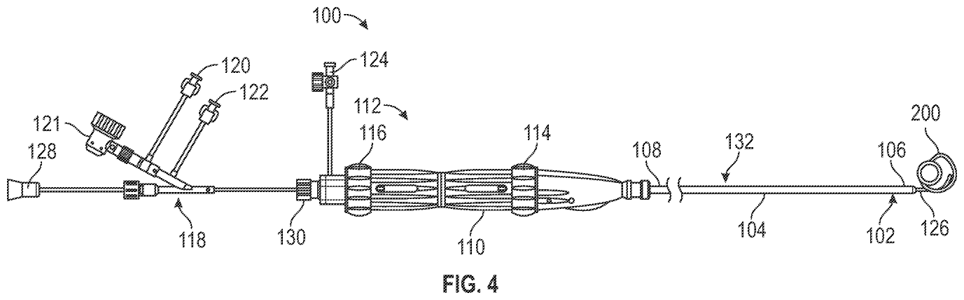

FIG. 4 shows an example of a delivery catheter 100 that may be utilized according to examples herein. The delivery catheter 100 may include an elongate shaft 104 having a distal end portion 102 ending in a distal tip 106. The distal tip 106 may include an aperture for the implant to pass through to deploy from the delivery catheter 100. The elongate shaft 104 may include a proximal portion 108 that may couple to a handle 110.

The handle 110 may be configured for a user to grip and manipulate to control the elongate shaft 104. For example, the handle 110 may be configured for a user to grip as the elongate shaft 104 is advanced distally into vasculature of a patient's body. The handle 110 may further be configured for a user to grip to rotate the elongate shaft 104 when positioned within the patient's vasculature. Rotation of the handle 110 may rotate the position of the distal tip 106 of the elongate shaft 104 to place the distal tip 106 in a desired configuration.

The handle 110 may further include a deflection mechanism 112 that may be utilized to deflect all or a portion of the elongate shaft 104, including one or more portions of the distal end portion 102. The deflection mechanism 112, for example, may be engaged with proximal portions of one or more tethers that may be configured to have a longitudinal force applied to the respective tether by the deflection mechanism 112 to deflect a portion of the elongate shaft 104.

The deflection mechanism 112, for example, may include one or more actuators 114, 116 that may be configured to be actuated by a user to move a respective tether. The actuators 114, 116, for example may comprise control knobs as shown in FIG. 4, or in examples may have other forms. The actuators 114, 116 may be configured to apply a longitudinal force to respective tethers within a tether channel to move the tether within the tether channel. The longitudinal force may result in a deflection of all or a portion of the distal end portion 102 of the elongate shaft 104. In other examples, other forms of deflection mechanisms may be utilized.

The delivery catheter 100 may further include various flushing ports 120, 122, 124 that may supply flush fluid to one or more lumens of the delivery catheter 100. The delivery catheter 100 may further include a hub assembly 118, which may include a suture lock assembly 121. The hub assembly 118 may be configured to control features of a system for deploying an anchoring device, which may include control of a pusher shaft 126 and a docking coil sleeve. A docking coil sleeve handle 128 may be utilized to control a position of a docking coil sleeve relative to the pusher shaft 126. The hub assembly 118 may be coupled to the handle 110 via a connector 130.

Features of the delivery catheter 100 and a delivery system that may be utilized in examples herein may be disclosed in International Patent Application PCT/US2020/036577, filed Jun. 8, 2020, and titled “Systems, Devices, and Methods for Treating Heart Valves,” and published as WO/2020/247907, and U.S. Patent Publication Nos. US2018/0318079, US2018/0263764, and US2018/0177594, which are all incorporated by reference herein in their entireties.

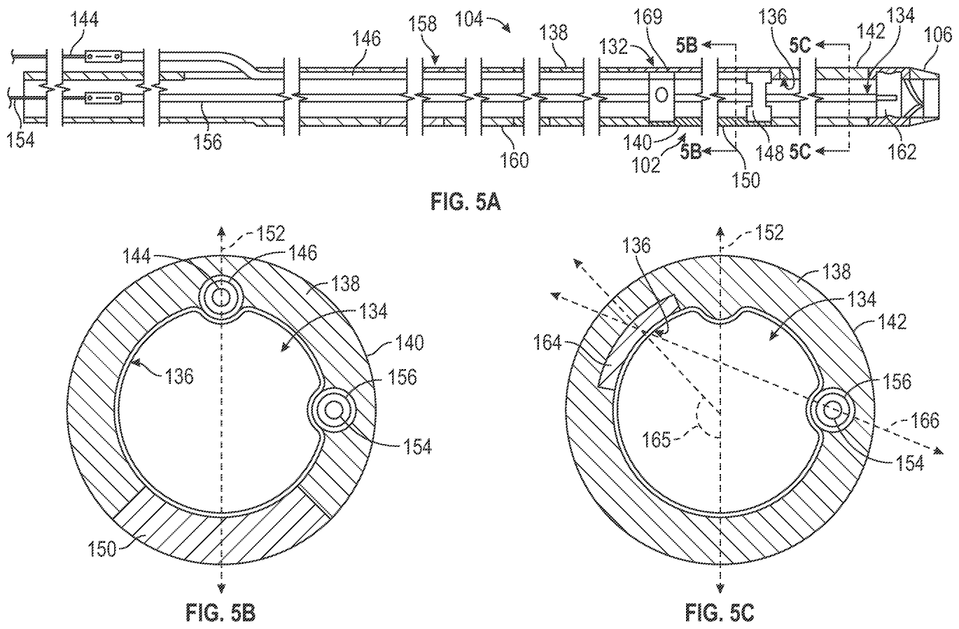

FIG. 5A illustrates a cross sectional view of the elongate shaft 104. The elongate shaft 104 may include an outer surface 132 that may be configured to slide within another catheter, such as the sheath 20 of the steerable guide sheath shown in FIG. 2. The elongate shaft 104 may be configured to bend, for example, to contour to a shape of a sheath 20 of a sheath catheter or other structure that the elongate shaft 104 may pass through. The elongate shaft 104 may have a cylindrical shape, or may have another shape in examples as desired.

The elongate shaft 104 may comprise a sheath that an implant such as an anchoring device, such as a docking coil, along with other components of the implant delivery system, may be configured to pass through. A docking coil sleeve be configured to pass through the elongate shaft 104. The elongate shaft 104 may include an interior lumen 134 that extends from the distal tip 106 of the elongate shaft 104 proximally to the proximal end of the elongate shaft 104. The interior lumen 134 may be configured for an implant such as an anchoring device, such as a docking coil, to be passed through and may further allow a docking coil sleeve to pass through. In examples, other components such as catheters or other devices may be passed through the interior lumen 134. The elongate shaft 104 may include an inner surface 136 that may face the interior lumen 134.

The elongate shaft 104 may include a wall 138 that may face the interior lumen 134. The wall 138 may be made of a flexible material that may allow all or a portion of the elongate shaft 104 to deflect in a desired manner.

The distal end portion 102 of the elongate shaft 104 may include one or more portions. The distal end portion 102, for example, may include a first flexible portion 140 and a second flexible portion 142 that is positioned distal of the first flexible portion 140.

The first flexible portion 140 may include a first tether 144 that may extend within a tether lumen 146 to a distal end of the first tether 144. The distal end may couple to a securing ring 148 or other anchoring point within the elongate shaft 104. The first flexible portion 140 may further include a first spine 150 that extends along the elongate shaft 104. The first spine 150 in examples may comprise a linear spine that extends along the longitudinal axis of the elongate shaft 104.

FIG. 5B illustrates a cross sectional view of the elongate shaft 104 along line 5B-5B in FIG. 5A, showing a cross sectional view of the first flexible portion 140. The first spine 150 may be positioned opposed circumferentially to the first tether 144 and the first tether lumen 146. The first spine 150, for example, may be positioned at a straight angle indicated by line 152 from the first tether 144. As such, a longitudinal force applied to the first tether 144 deflects the first flexible portion 140 in a plane along line 152. The first tether 144 may be positioned across the interior lumen 134 from the first spine 150.

The first spine 150 and the first tether 144 may each be embedded in the body of the elongate shaft 104. The first spine 150 may include a material that has a greater stiffness and higher durometer than an adjacent portion of the wall 138. The adjacent portions of the wall 138 may be adjacent circumferentially with respect to the first spine 150.

The first flexible portion 140 may include a second tether 154 and a second tether lumen 156 that extends through the first flexible portion 140. The second tether 154 may be positioned orthogonal with the first tether 144 and the first spine 150. The second tether 154 may be orthogonal from the first tether 144 in a clockwise direction when facing a proximal direction of the elongate shaft 104, as shown in FIG. 5B for example.

Referring to FIG. 5A, the first flexible portion 140 may be positioned between the second flexible portion 142 and a portion 158 of the elongate shaft 104 positioned proximate the first flexible portion 140. The portion 158 may include a wall 160 that has a greater stiffness and higher durometer than the first flexible portion 140, thus allowing the first flexible portion 140 to deflect relative to the portion 158 when a longitudinal force is applied to the first tether 144.

The second flexible portion 142 may be positioned distal of the first flexible portion 140 and proximal of the distal tip 106 of the elongate shaft 104. In examples, the second flexible portion 142 may include the distal tip 106.

FIG. 5C illustrates a cross sectional view of the second flexible portion 142. The second flexible portion may include the second tether 154 that may extend distally from the first flexible portion 140 shown in FIG. 5B. The second tether lumen 156 may extend distally from the first flexible portion 140 and the second tether 154 may extend within the second tether lumen 156. The second tether lumen 156 may have a distal end that may couple to a securing device such as a securing ring 162 as shown in FIG. 5A.

The second tether 154 may be positioned axially in line in the second flexible portion 142 with its position in the first flexible portion 140. Thus, as shown in FIGS. 5B and 5C, the second tether 154 may be in the same circumferential position. The second tether 154 may be positioned orthogonal relative to the first tether 144 and the first spine 150.

The second flexible portion 142, however, may include a second spine 164 such as a second linear spine that is positioned offset from the circumferential position of the first linear spine 150 shown in FIG. 5B. The second linear spine 164 may be positioned non-orthogonal and non-parallel relative to the first linear spine 150, as shown in the relative positions between the first linear spine 150 and the second linear spine 164 in FIGS. 5B and 5C. The second linear spine 164, for example, may be positioned at an obtuse angle 165 relative to the first linear spine 150 as shown in the relative positions in FIGS. 5B and 5C. Such an obtuse angle 165 may comprise a range between 91 degrees and 179 degrees in examples. In examples, the second linear spine 164 may be positioned at an acute angle relative to the first linear spine 150.

The second linear spine 164 and the second tether 154 may each be embedded in the body of the elongate shaft 104. The second tether 154 in examples may comprise a pull tether configured to be retracted proximally to deflect the second flexible portion 142. The first tether 144 in examples may comprise a pull tether configured to be retracted proximally to deflect the first flexible portion 140.

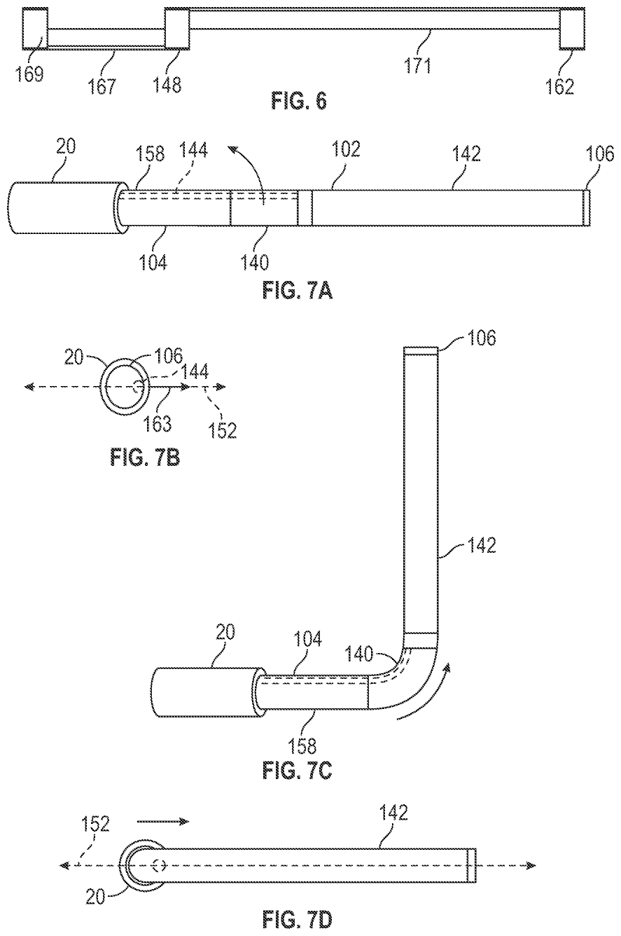

The second linear spine 164 may have a proximal portion that couples to a distal portion of the first linear spine 150, with the second linear spine 164 offset from the circumferential position of the first linear spine 150. FIG. 6 for example, illustrates a cross sectional representation of spines for a delivery catheter. A proximal spine 167, which may correspond to the first linear spine 150 may have a distal portion that couples to a securing device such as securing ring 148, and may have a proximal portion that couples to a securing device such as securing ring 169. The distal portion of the proximal spine may couple to the proximal portion of a distal spine 171 via the securing ring 148 or another manner of coupling. The distal spine 171 may correspond to the second linear spine 164. The distal spine 171 may have a distal portion that couples to the securing ring 162. The spines accordingly may comprise a unitary body in examples, with the spines coupled together. The spines shown in FIG. 6 are positioned parallel with each other. The spines 150, 164 shown in FIGS. 5B and 5C, however, are positioned non-parallel and non-orthogonal with each other.

Referring back to FIG. 5C, the second linear spine 164 may be positioned non-parallel from the second tether 154. Further, the second linear spine 164 may be positioned non-orthogonal from the second tether 154. The second linear spine 164, in examples, may be positioned at an obtuse angle circumferentially from the second tether 154, which may comprise a range between 91 degrees and 179 degrees in examples. The second linear spine 164 may be positioned on an opposite side of the wall 138 from the second tether 154 and may be positioned at a circumferential orientation with respect to the second tether 154 that is between a circumferentially opposed position and the circumferential position of the first tether 144 that is shown in FIG. 5B. The second linear spine 164 may be positioned at an acute angle relative to the first tether 144 as shown in FIGS. 5B and 5C. The second linear spine 164 accordingly may be positioned closer to the second tether 154 in a clockwise direction when facing proximal than in a counterclockwise direction.

The relative orientation of the second tether 154 and the second linear spine 164 shown in FIG. 5C may allow the second flexible portion 142 to deflect in a direction that is non-orthogonal and non-parallel with the plane (represented by line 152) that the first flexible portion 140 may deflect in, upon a longitudinal force being applied to the second tether 154. The non-parallel position of the second linear spine 164 and the second tether 154, for example, may allow the second flexible portion 142 to deflect in a direction defined by line 166 in FIG. 5C. The line 166 for example may extend between the second linear spine 164 and the second tether 154, and as shown in FIG. 5C is non-parallel and non-orthogonal with the line 152 (which may represent the plane of deflection of the first flexible portion 140).

The direction that the second flexible portion 142 is configured to deflect in may be obtuse relative to the direction of deflection of the first flexible portion 140. As such, when the first flexible portion 140 is configured to deflect upward in a plane as shown in FIG. 5B, the second flexible portion 142 may be configured to deflect downward and out of the plane due to the orientation of the second linear spine 164 and the second tether 154 shown in FIG. 5C.

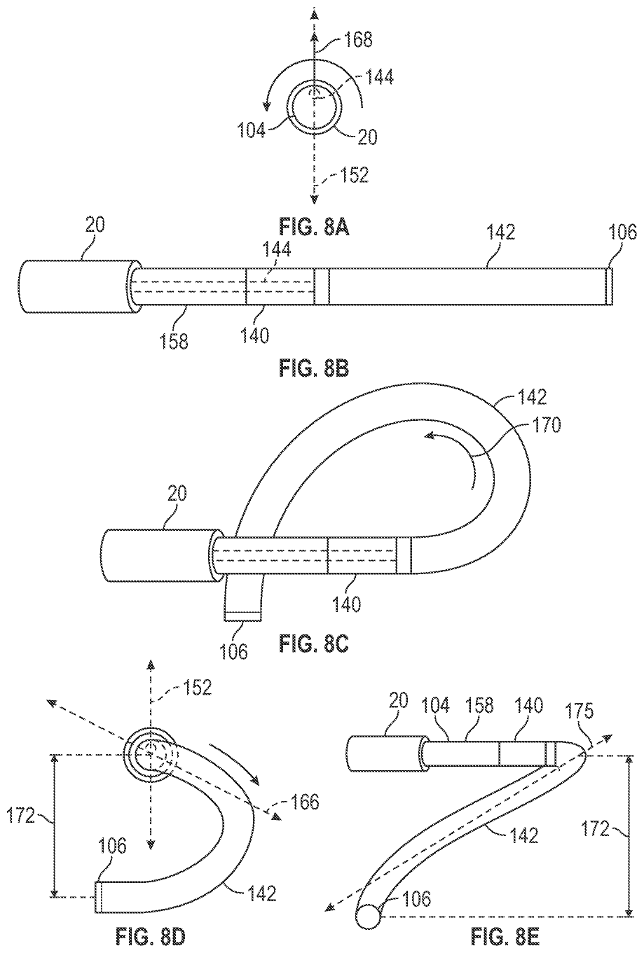

FIGS. 7A-8E for example, illustrate exemplary deflection of the distal end portion 102, including the first flexible portion 140 and the second flexible portion 142. The position of the first tether 144 is shown in dashed lines for reference. FIGS. 7A-7D illustrate an exemplary deflection of the first flexible portion 140.

Referring to FIG. 7A, the first flexible portion 140 and the second flexible portion 142 are shown to extend linearly from the guide sheath 20. The first flexible portion 140 may be configured to deflect in a plane that the first tether 144 and the first spine 150 extend in, and represented by line 152 in FIG. 5B. The direction of deflection may be towards the first tether 144 upon the first tether 144 being retracted in a proximal longitudinal direction. FIG. 7B, for example, illustrates the plane along line 152 and an arrow 163 representing the direction of deflection.

FIG. 7C, for example, illustrate the first flexible portion 140 having been deflected in the direction of the arrow 163 in FIG. 7B. The first tether 144 may be retracted to deflect the first flexible portion 140. The first flexible portion 140 may deflect in the plane along line 152 shown in FIGS. 7B and 7D. The second flexible portion 142 accordingly may be deflected to be positioned at an angle relative to the proximal portion 158 of the elongate shaft 104. The first flexible portion 140 may be configured to deflect up to a 90 degree angle in the plane defined by line 152, or up to a 180 degree angle in examples if desired.

FIGS. 8A-8E illustrate a rotation of the elongate shaft 104 and an exemplary deflection of the second flexible portion 142. As shown in FIG. 8A, the elongate shaft 104 may be rotated relative to the guide sheath 20 and such rotation may be in a counterclockwise or clockwise direction when facing proximally, yet in FIG. 8A a counterclockwise rotation is shown. The rotation may be 90 degrees to position the first tether 144 orthogonal from the position shown in FIG. 7B. In examples, other degrees of rotation may be utilized.

The first tether 144 accordingly may be positioned upward in FIG. 8A and the first flexible portion 140 may be configured to deflect in an upward direction 168 in the plane defined by line 152. FIG. 8B illustrates the resulting orientation of the first tether 144 in the position shown in FIG. 8A.

FIGS. 8C and 8D illustrate an exemplary direction of deflection of the second flexible portion 142 with the first flexible portion 140 in the orientation shown in FIG. 8B. The second flexible portion 142 may deflect in a direction that is non-orthogonal and non-parallel with the plane defined by line 152 that the first flexible portion 140 is configured to deflect in. The second tether 154 may be retracted to deflect the second flexible portion 142. The relative orientations of the directions of deflection are shown in FIG. 8D.

As shown in FIGS. 8C and 8D, the second flexible portion 142 may be configured to deflect to form a curve extending proximally upon a longitudinal force being applied to the second tether 154. As such, the degree of deflection 170 of the second portion 142 may be greater than 90 degrees in examples, and may be greater than 180 degrees in examples, as shown in FIG. 8C. The curve of the second flexible portion 142 may position the distal tip 106 of the second flexible portion 142 at an angle that differs from the angle shown in FIG. 8B, and may be orthogonal from the orientation shown in FIG. 8B. In examples, other angles may be formed by the deflection of the second flexible portion 142.

FIG. 8E illustrates a side view of the elongate shaft 104 at a view that is rotated 90 degrees from the view in FIG. 8D. The curve of the second flexible portion 142 is shown to extend in a plane 175 that is non-orthogonal and non-parallel with the plane defined by line 152 shown in FIG. 8D that the first flexible portion 140 is configured to deflect in.

In the side view of FIG. 8E, the distal tip 106 is shown to extend in a plane that is parallel and offset with a plane that the first flexible portion 140 extends in. The distance between the planes may be defined by the height 172 marked in FIGS. 8D and 8E. The distal tip 106 may be positioned beneath a portion of the elongate shaft 104 and may be directed transverse to a direction of the distal tip 106 shown in FIG. 8B. The distal tip 106 accordingly may be oriented in a different direction and may be at a different height than shown in FIG. 8B.

The deflection of the second flexible portion 142 may form a height 172 between a proximal portion of the second flexible portion 142 and a distal portion of the second flexible portion 142 that may include the distal tip 106. The height 172 may further be between the distal tip 106 and the first flexible portion 140, or the proximal portion 158 of the elongate shaft 104, or the guide sheath 20 as shown in FIG. 8E. The height 172 may allow an implant to be deployed from the distal tip 106 at a lower height relative to the proximal portion of the second flexible portion 142. As such, during a procedure, the height 172 may be utilized to position the distal tip 106 in a direction that is ventricular with regard to the proximal portion of the second flexible portion 142, and thus may position the distal tip 106 in a more ventricular direction that may be proximate a commissure of the mitral valve in examples. The first flexible portion 140 accordingly may be positioned in an atrium and the height 172 may be in a ventricular direction. Further, the curve of the second flexible portion 142 may be planar with respect to the mitral plane to allow the implant to deploy from the distal tip 106 in the mitral plane.

The curve of the second flexible portion 142 as shown in FIGS. 8C-8E may be counterclockwise with respect to the mitral annulus when viewing the second flexible portion 142 in a direction towards the ventricle from the atrium. Such a direction of curvature may allow an anchoring device, such as a docking coil, to deploy in a counterclockwise curvature with respect to the mitral annulus when viewing the second flexible portion 142 in a direction towards the ventricle from the atrium. In examples, another direction of curvature (e.g., clockwise when viewing the second flexible portion 142 in a direction towards the ventricle from the atrium) may be utilized.

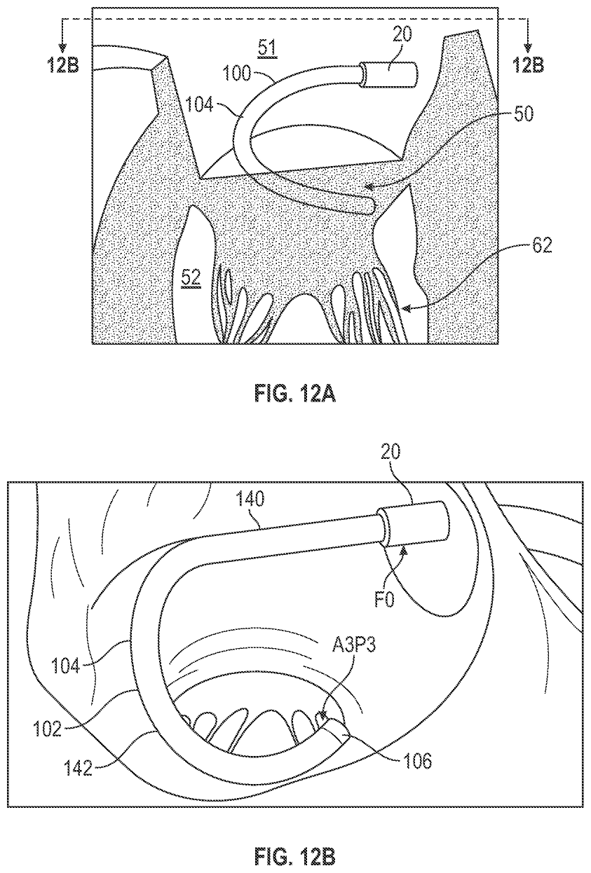

The configuration of the elongate shaft 104 shown in FIGS. 8C-8E may be utilized to deploy an anchoring device to the mitral valve or another location within a patient's body as desired. The configuration of the elongate shaft 104 shown in FIGS. 8C-8E for example, may correspond to the position of the elongate shaft 104 shown in FIGS. 12A and 12B for example.

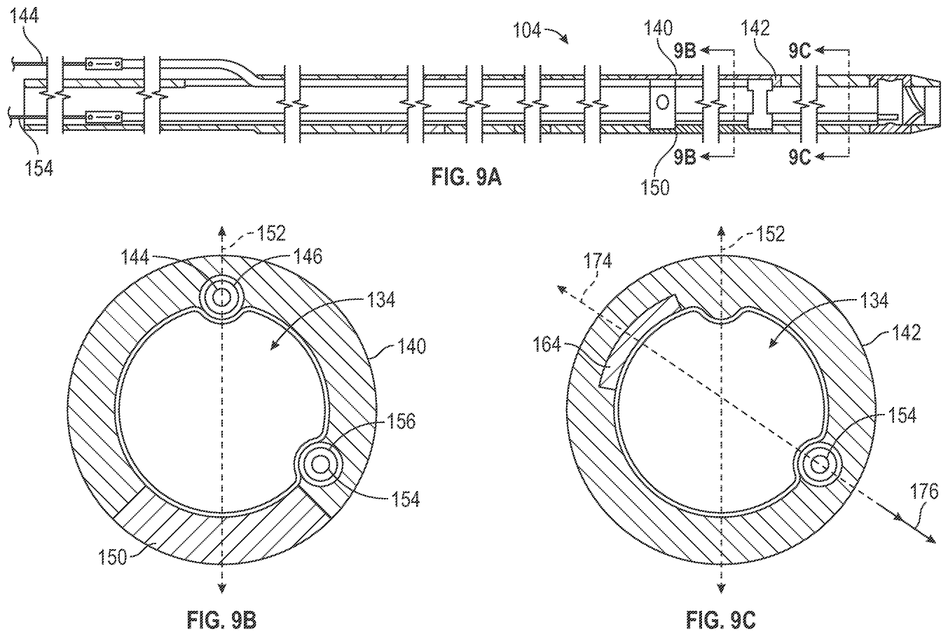

FIGS. 9A-9C illustrates an example of the elongate shaft 104 in which the orientation of the second tether 154 relative to the second spine 164 differs from the orientation shown in FIG. 5C. In the example of FIGS. 9A-9C, the second tether 154 is positioned opposed circumferentially to the second linear spine 164. As such, the second flexible portion 142 is configured to deflect about a plane defined by line 174, in the direction 176. The second tether 154 may be positioned at a straight angle relative to the second linear spine 164.

The second linear spine 164 is positioned non-orthogonal and non-parallel relative to the first linear spine 150 shown in FIG. 9B. The second flexible portion 142 is configured to deflect in the direction 176 that is non-orthogonal and non-parallel with the plane defined by line 152 that the first flexible portion 140 is configured to deflect in, upon a longitudinal force being applied to the second tether 154. The direction 176 may be obtuse relative to a direction of deflection of the first flexible portion 140.

The second tether 154 may be positioned at an obtuse angle relative to the position of the first tether 144, as shown in FIGS. 9B and 9C. Further, in examples, the second linear spine 164 may be positioned at an obtuse angle relative to the position of the first linear spine 150. The second linear spine 164 may be positioned at an acute angle relative to the first tether 144.

The deflection of the second flexible portion 142 may result in a similar configuration as shown in FIGS. 8C-8E, with a curve extending proximally and a height 172 being formed between the distal tip 106 and the proximal portion of the second flexible portion 142 and the first flexible portion 140.

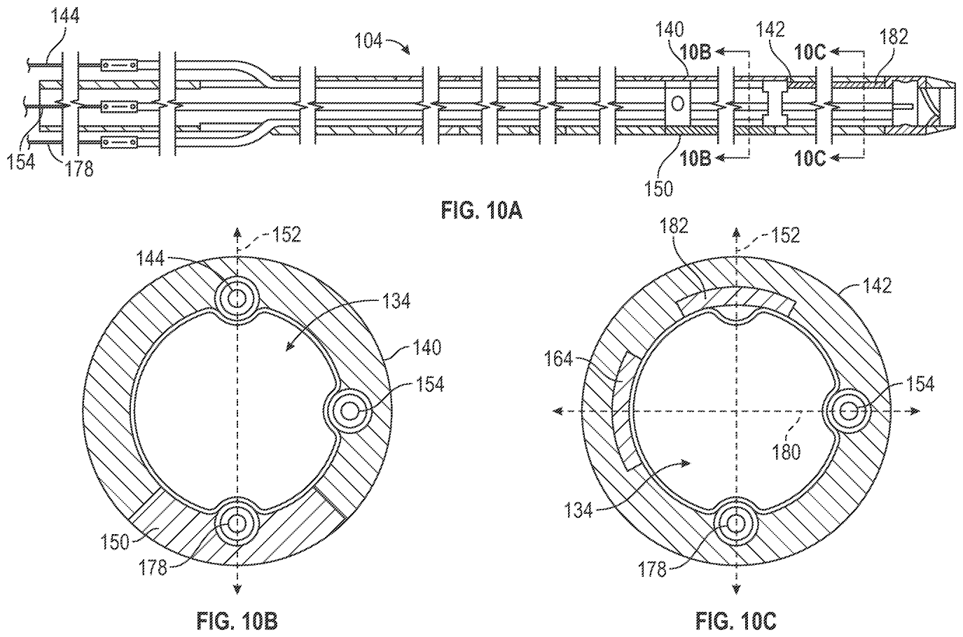

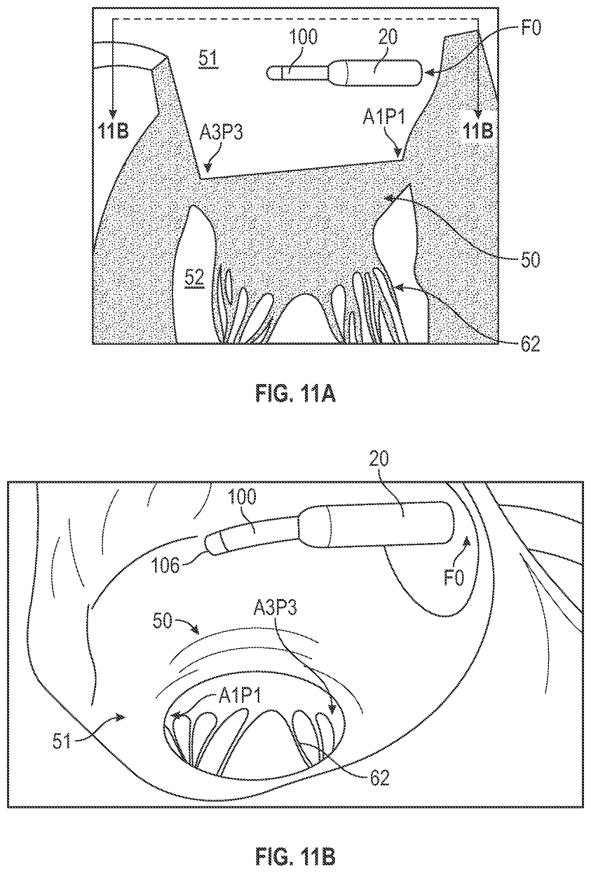

FIGS. 10A-10C illustrate an example in which the elongate shaft 104 includes a third tether 178. The third tether 178 may extend along the elongate shaft 104 and may extend to the second flexible portion 142. For example, as shown in the cross sectional view of FIG. 10B, the first flexible portion 140 may include the first tether 144 positioned opposed circumferentially to the first linear spine 150. The first flexible portion 140 may be configured to deflect in a plane defined by line 152 upon a longitudinal force being applied to the first tether 144. The second tether 154 may extend along the first flexible portion 140, and may extend at a position that is orthogonal with respect to the first tether 144 and the first linear spine 150.

The third tether 178 may extend through the first flexible portion 140 at a position that is circumferentially opposed to the first tether 144, and that may be orthogonal to the position of the second tether 154. The first flexible portion 140 may be configured to deflect in the plane defined by line 152. In examples, the third tether 178 may extend through the first spine 150 or may otherwise be positioned to allow the third tether 178 to pass through the first flexible portion 140.

FIG. 10C illustrates an example of the second flexible portion 142 illustrating a position of the second linear spine 164 relative to the second tether 154. The second linear spine 164 may be positioned circumferentially opposed to the second tether 154 and may be positioned orthogonal relative to the position of the first linear spine 150 and the first tether 144 as shown in FIG. 10B. The second tether 154 may be positioned orthogonal relative to the first tether 144. As such, the second flexible portion 142 may be configured to deflect in a plane defined by line 180 upon a longitudinal force being applied to the second tether 154. The plane may be orthogonal to the plane that the first flexible portion 140 deflects in, defined by line 152.

The third tether 178 may be utilized to deflect the second flexible portion 142 in a direction that is away from the direction of deflection of the first flexible portion 140. As such, the third tether 178 may extend within the second flexible portion 142 and may be positioned opposed circumferentially relative to the first tether 144 shown in FIG. 10B. The third tether 178, as such, when pulled with a longitudinal force applied to the third tether 178 may deflect the second flexible portion 142 in a direction that is away from the direction of deflection of the first flexible portion 140. The third tether 178 may be configured to allow the second flexible portion 142 to deflect along the plane defined by line 152, yet in an opposite direction as the first flexible portion 140. The third tether 178 in examples may comprise a pull tether configured to be retracted proximally to deflect the second flexible portion 142.

In examples, the second flexible portion 142 may include a third linear spine 182 that may be positioned circumferentially opposed to the third tether 178 and orthogonal from the second tether 154. The third linear spine 182 may be positioned in line with the first tether 144 shown in FIG. 10B. In examples, the third linear spine 182 may be excluded.

In operation, the first flexible portion 140 may be configured to flex in a similar manner as shown in FIGS. 7A-7D. The second flexible portion 142 may be configured to form a curve in a plane that is orthogonal to the plane of the first flexible portion 140 upon the second tether 154 being retracted. The curve may extend proximally. The third tether 178 may be retracted to cause the second flexible portion 142 to produce a height, and result in a configuration that is similar to the configuration shown in FIGS. 8C-8E. The third tether 178 may allow a user to control the height of the resulting curve based on the amount of tension placed upon the third tether 178.

Accordingly, the second flexible portion 142 may be configured to deflect in a direction that is obtuse relative to a direction of deflection of the first flexible portion 140 upon a longitudinal force between applied to both the second tether 154 and the third tether 178. The second flexible portion 142 may extend in a plane that is non-orthogonal and non-parallel with the plane defined by line 152 upon a longitudinal force being applied to both the second tether 154 and the third tether 178.

The examples of delivery catheters, elongate shafts, and distal end portions of elongate shafts may be utilized to deploy an anchoring device, which may comprise a docking coil in examples. The features disclosed herein may comprise a system for delivering an implant to a portion of a patient's body. Various sheath and delivery catheter designs may be used to effectively deploy an anchoring device at an implantation site. For example, for deployment at the mitral position, the delivery catheter can be shaped and/or positioned to point towards commissure A3P3, so that a coil anchor deployed from the catheter can more easily enter the left ventricle and encircle the chordae 62 during advancement. However, while the various exemplary examples of the disclosure described below are configured to position the distal opening of the delivery catheter at commissure A3P3 of the mitral valve, in other examples, the delivery catheter can approach the mitral plane to point to, and the anchoring device can be advanced through, commissure A1P1 instead. In addition, the catheter can bend either clockwise or counter-clockwise to approach either commissure of the mitral valve or a desired commissure of another native valve, and the anchoring device can be implanted or inserted in a clockwise or counter-clockwise direction (e.g., coils/turns of the anchoring device can turn in a clockwise or counter-clockwise direction depending on how the anchoring device will be implanted).

FIGS. 11A-12B, for example, illustrate a method of positioning using the examples of elongate shafts of delivery catheters disclosed herein. The positioning may include locating the distal tip 106 of the delivery catheter 100 at a commissure of the mitral valve and may include placing the curve of the distal end portion 102 in a plane with the mitral annulus. FIGS. 11A-12B, for example illustrate a method of positioning the delivery catheter 100 to deliver an implant such as an anchoring device to a native valve. The anchoring device may comprise a docking device, such as a docking coil, as disclosed herein.

The delivery catheter 100 may be advanced to a position within the patient's body. The delivery catheter 100 may comprise any example of delivery catheter disclosed herein. The delivery catheter 100 may deliver and implant an implant in the form of an anchoring device (which can be the same as or similar to other anchoring devices described herein) at a native valve of a patient (e.g., at the native mitral valve 50 of a patient using a transseptal technique).

FIG. 11A is a cutout view of the left atrium of a patient's heart that illustrates a sheath 20 (e.g., a guide sheath or transseptal sheath) of a sheath catheter passing through the interatrial septum, which can happen at the fossa ovalis (FO), and into the left atrium, and a delivery catheter 100 extending from the sheath 20.

FIG. 11B illustrates the guide sheath 20 and the delivery catheter 100 in the position shown in FIG. 11A from a view looking down at the mitral valve 50 from the left atrium 51 (i.e., from a view taken along the line 11B-11B in FIG. 11A). Referring to FIG. 11A, the sheath 20 may enter the left atrium such that the sheath may be substantially parallel with the plane of the mitral valve 50. The guide sheath 20 can take any suitable form, such as, for example, any form described in the present application.

In some examples, the sheath 20 can be actuated or steerable as a steerable guide sheath so that the sheath 20 can be positioned or bent until it makes an angle (e.g., a 30-degree angle or an approximately 30-degree angle) with respect to the septum and/or FO wall. In some examples, the angle orientation (e.g., 30-degree angle orientation) can be adjusted or controlled by rotating or further actuating the sheath 20, and can be adjusted to better control the orientation at which the delivery catheter 100 enters the left atrium. In other examples, the deflection angle of the sheath 20 relative to the septum and/or FO can be either more or less than 30 degrees, depending on each situation, and in some applications, can even be oriented at or bent to be 90 degrees relative to the septum and/or FO. In certain examples, the deflection angle of the sheath can be moved between about 0 degrees and about 90 degrees, such as, for example, between about 5 degrees and about 80 degrees, such as between about 10 degrees and 70 degrees, such as between about 15 degrees and about 60 degrees, such as between about 20 degrees and about 50 degrees, such as between about 25 degrees and about 40 degrees, such as between about 27 degrees and about 33 degrees.

Referring to FIGS. 12A and 12B, after the outer sheath or guide sheath 20 passes through the septum and/or FO and is placed in a desired position, the delivery catheter 100 exits and extends from the sheath 20. The delivery catheter 100 may be moved distally from the sheath 20 such that the delivery catheter 100 in such a configuration extends from the guide sheath 20 with a straightened shape. In examples, the distal end portion 102 of the delivery catheter 100 may initiate deflection, however, the delivery catheter 100 may extend in a straightened shape for some distance in the atrium. The delivery catheter 100 may be positioned in a desired location via the extension of the delivery catheter 100 from the guide sheath 20 and via deflection of the guide sheath 20 to a desired amount. The guide sheath 20, for example, may be deflected in a ventricular direction to angle the delivery catheter 100 in such a direction.

With the delivery catheter 100 extending in the left atrium 51, the first flexible portion 140 and/or second flexible portion 142 may be deflected to position the distal tip 106 of the elongate shaft in the desired location relative to the mitral annulus.

In examples, a method may include inserting the delivery catheter 100 into the left atrium 51 with the first flexible portion 140 configured to deflect upward in a direction that is away from the mitral annulus (e.g., an atrial direction). Such an orientation may be shown in FIG. 8A, with the first tether 144 configured to deflect the first flexible portion 140 in a direction away from the mitral annulus. The first flexible portion 140, however, may not be deflected in such a direction, and the second flexible portion 142 rather may be deflected as shown in FIGS. 8C-8E with the second flexible portion 142 extending in a curve downward in a ventricular direction towards the mitral valve. The curves shown in FIGS. 8D and 8E, for example, may extend downward in a ventricular direction, with the height 172 shown in FIGS. 8D and 8E extending in in a ventricular direction towards the mitral valve.

The resulting configuration of the distal end portion 102 may extend to the commissure of the mitral valve, which may comprise the A3P3 commissure as shown in FIG. 12B for example. The elongate shaft 104 may be positioned within the atrium and the second flexible portion 142 may be deflected to a commissure of the patient's mitral valve. The curve of the second flexible portion 142 may extend in the plane of the mitral annulus for deployment of the anchoring device at the commissure of the mitral valve.

The distal tip 106 may be positioned below the commissure point and may extend into the ventricle in examples if desired. The delivery catheter 100 may deflect downward until the circular/curved planar portion of the distal end portion 102 nears the plane of the mitral valve 50, which is generally about 30 to 40 mm below the FO wall. In some situations, however, the plane of the mitral valve may be less than 30 mm below the FO or more than 30 mm below the FO. In certain examples, the delivery catheter 100 is configured to extend 60 mm or less from the outer sheath, such as, for example, 50 mm or less, such as 45 mm or less, such as 40 mm or less, such as 35 mm or less, such as 30 mm or less, such as 25 mm or less, such as 20 mm or less. In some examples, the maximum extension of the delivery catheter 100 from the exterior sheath is between about 20 mm and about 60 mm, such as, for example, between about 25 mm and about 50 mm, such as between 30 mm and about 40 mm.

The lower curved portion of the distal end portion 102 shape may be lowered to or near the level of the annulus, the lower curved portion can be parallel or nearly parallel (e.g., planar or nearly planar) with a plane of the annulus, or the lower curved can be slightly upwardly angled relative to the plane of the annulus.

In examples, additional deflections of the catheter 100 may be utilized. For example, upon entry into the left atrium 51, the first flexible portion 140 may be oriented as shown in FIG. 7B, with the first flexible portion configured to deflect in a plane that is parallel and offset with the plane of the mitral annulus. In such a configuration, the second flexible portion 142 may be deflected partially or fully to extend in a ventricular direction towards the mitral valve. As such, the distal tip 106 may extend in a downward ventricular direction towards the left ventricle.

With the second flexible portion 142 deflected partially or fully, the first flexible portion 140 may be deflected in a plane parallel with the plane of the mitral annulus, similar to the deflected configuration shown in FIG. 7C. The second flexible portion 142 in such a configuration, however, may remain extending in a ventricular direction with the distal tip 106 positioned proximate a commissure of the mitral valve. The deflection of the first flexible portion 140 and/or the second flexible portion 142 may be adjusted as desired to position the distal tip 106 at the desired commissure of the mitral valve, for example the A3P3 commissure. Further a rotation of the delivery catheter 100 may be utilized to position the distal tip 106 at a desired location relative to the A3P3 commissure.

In a step in the method, an anchoring device, such as a docking coil may be partially extended out of the distal tip 106 to be positioned within the ventricle and exterior of the mitral valve leaflets. Such a procedure may cause the anchoring device to hook a portion of the mitral valve leaflet to maintain a position of the distal tip 106 of the delivery catheter 100. In examples, a step of extending the docking coil partially may be excluded.

With the distal tip 106 at a desired location relative to the A3P3 commissure, the deflection of the first flexible portion 140 may be returned towards a straightened configuration and the delivery catheter 100 may be rotated in the direction shown in FIG. 8A to result in the second flexible portion 142 being in the orientation shown in FIGS. 8C-8E. As such, the resulting second flexible portion 142 may be in the configuration shown in FIGS. 12A and 12B and configured for deployment of the anchoring device in the mitral plane. In such a configuration, the curve of the second flexible portion 142 extending in the ventricular direction may assist the distal tip 106 to not come loose from its position at the A3P3 commissure when the delivery catheter 100 is rotated in the direction shown in FIG. 8A.

The curve of the second flexible portion 142 being configured to extend in the ventricular direction accordingly comprises an improvement upon a configuration in which the first flexible portion and second flexible portion would deflect in orthogonal planes. In a configuration in which the first flexible portion and the second flexible portion deflect in orthogonal planes, a torque may be asserted against the second flexible portion when the delivery catheter is rotated in the direction shown in FIG. 8A. Such a torque may result in the distal tip coming loose from its position at the A3P3 commissure undesirably.

The resulting configuration shown in FIGS. 12A and 12B may result whether the configurations of elongate shafts 104 shown in the various examples of FIGS. 5A-10C are utilized.

With the delivery catheter 100 in the configuration shown in FIGS. 12A and 12B, the anchoring device may be deployed to the implantation site. The anchoring device may have a variety of forms, examples of which may be shown in International Patent Application PCT/US2020/036577, filed Jun. 8, 2020, and titled “Systems, Devices, and Methods for Treating Heart Valves,” and published as WO/2020/247907, which is incorporated by reference herein in its entirety. The implant in the form of an anchoring device may be deployed from the interior lumen of the delivery catheter 100 to the implantation site within the patient's body. In an example in which the implant comprises a docking coil, the docking coil may be deployed around leaflets of the patient's mitral valve.

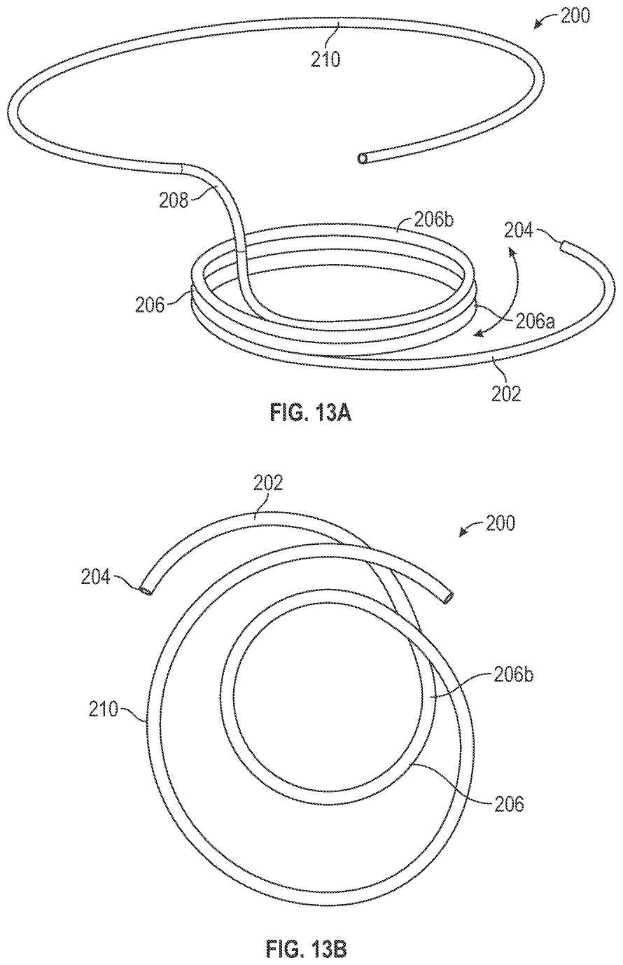

FIGS. 13A and 13B illustrate an example of an anchoring device that may be utilized according to examples herein. The anchoring device, for example, may comprise a docking coil 200 that may be configured to dock with an implant within a portion of a patient's body.

The docking coil 200 may include one or more turns that may be utilized for implantation and/or stabilization within the patient's body. The docking coil 200, for example, may include an encircling or leading turn 202 that may extend to a distal or leading tip 204 of the docking coil 200. The encircling or leading turn 202 may be configured to encircle native structure of the patient's heart during deployment, for example, native valve leaflets and chordae that are encircled during implantation of the docking coil 200.

A proximal portion of the encircling or leading turn 202 may couple to one or more functional turns 206. The functional turns 206 may be shaped into a coil with the turns 206 stacked upon each other along a central axis of the docking coil 200. The functional turns 206 may include one or more lower turns 206a and may include one or more upper turns 206b. The lower turns 206a in examples may be configured to be positioned on the ventricular side of the mitral valve and the upper turns 206b in examples may be configured to be positioned on the atrial side of the mitral valve. As such, the mitral valve in examples may be configured to be positioned between functional turns 206 of the docking coil 200.

In examples, the lower turns 206a and upper turns 206b may be both configured to be positioned on the ventricular side of the mitral valve, and encircling the mitral valve leaflets and other native structures such as chordae.

In examples, a transition curve 208 may couple to a proximal portion of the functional turns 206 and may extend to a stabilization turn 210 that may have a larger diameter than the functional turns 206 and may be configured to be positioned at the atrial side of the mitral valve. The transition curve 208 may extend in an axial dimension and may be configured to pass through the commissure of the mitral valve to transition between the functional turns 206 and the stabilization turn 210.

FIG. 13B illustrates a top view of the docking coil 200 shown in FIG. 13A. In examples, the configuration of the docking coil 200 may be varied as desired. Features of a docking coil that may be utilized in examples herein may be disclosed in International Patent Application PCT/US2020/036577, filed Jun. 8, 2020, and titled “Systems, Devices, and Methods for Treating Heart Valves,” and published as WO/2020/247907, which is incorporated by reference herein in its entirety.

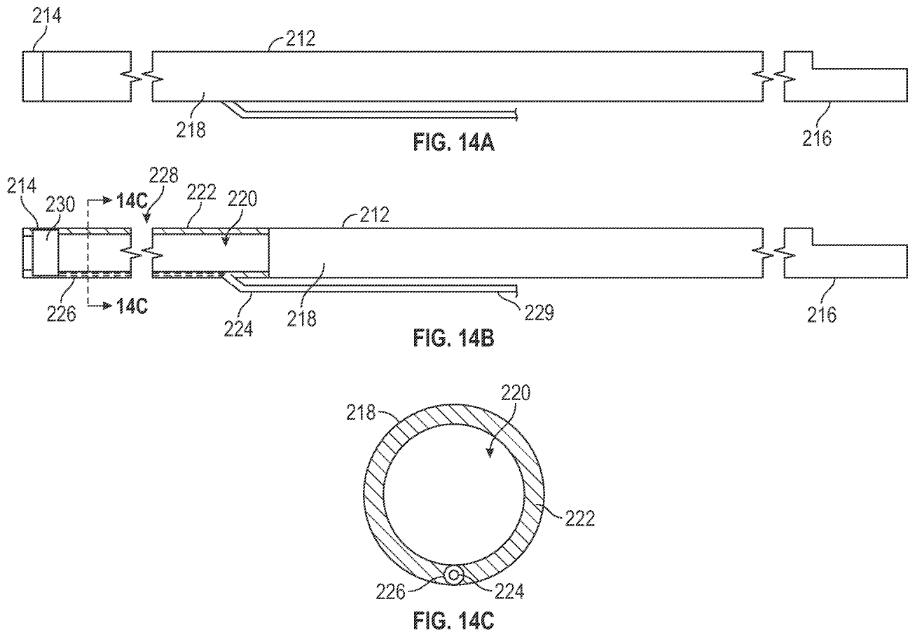

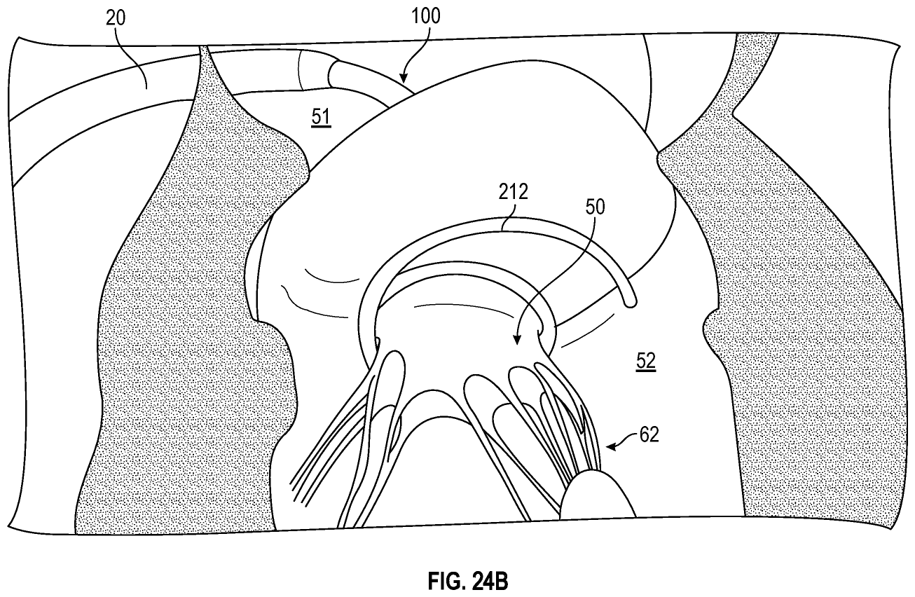

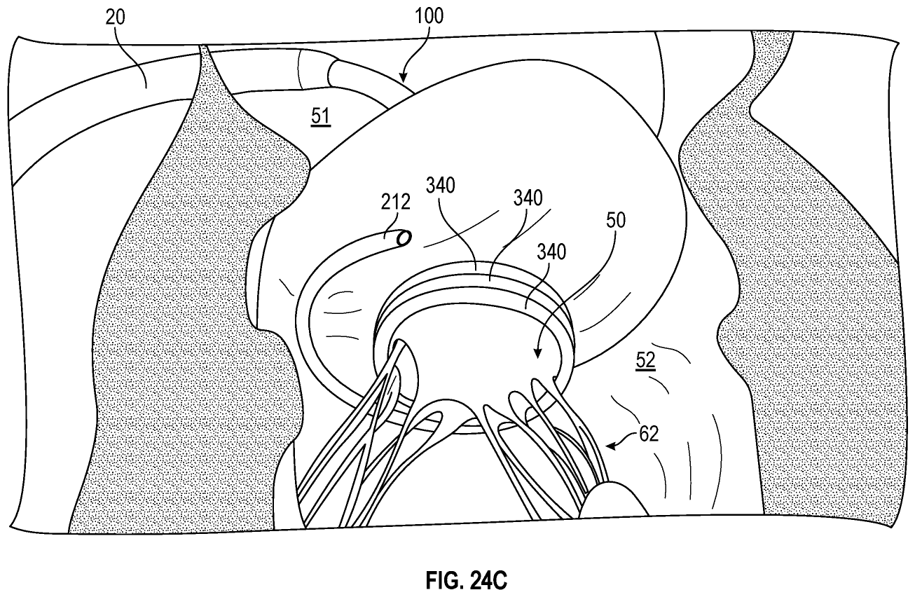

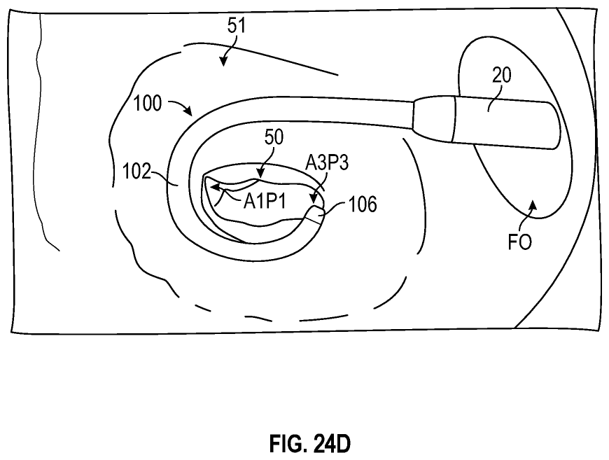

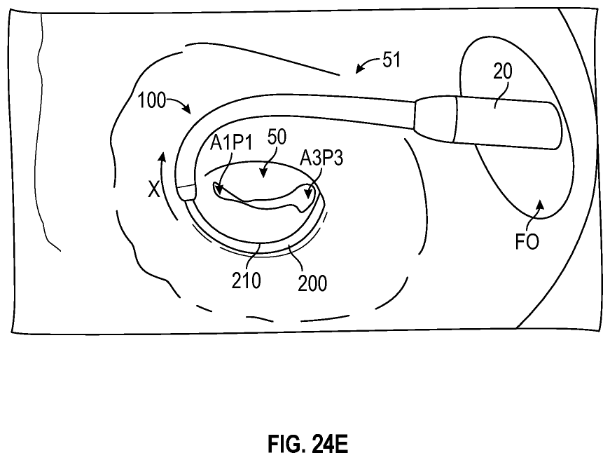

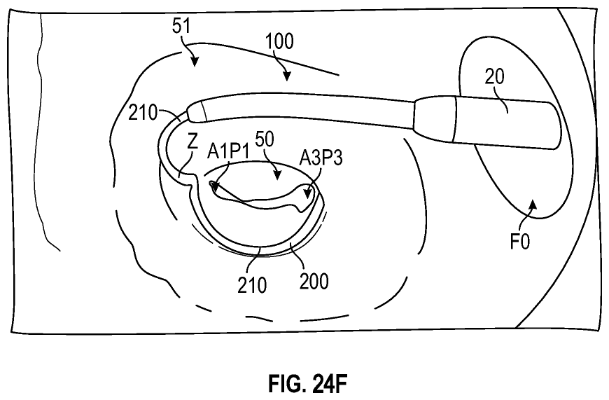

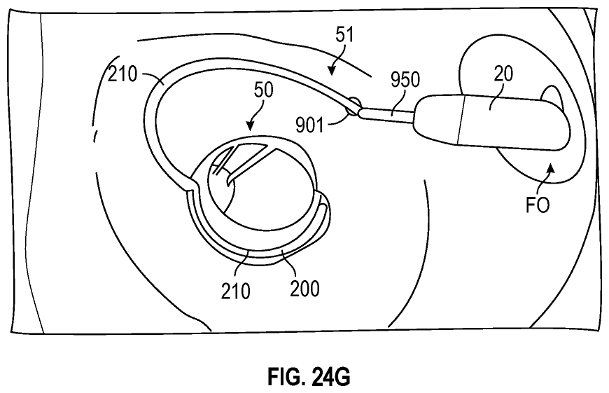

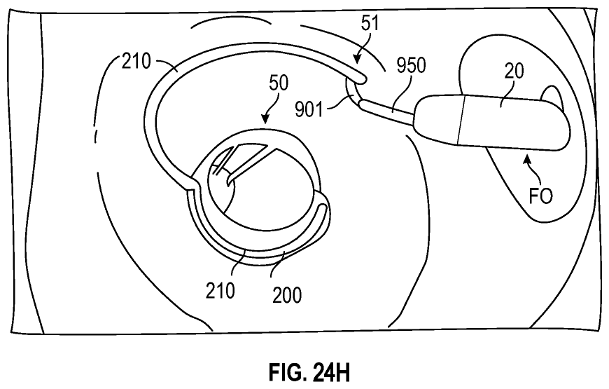

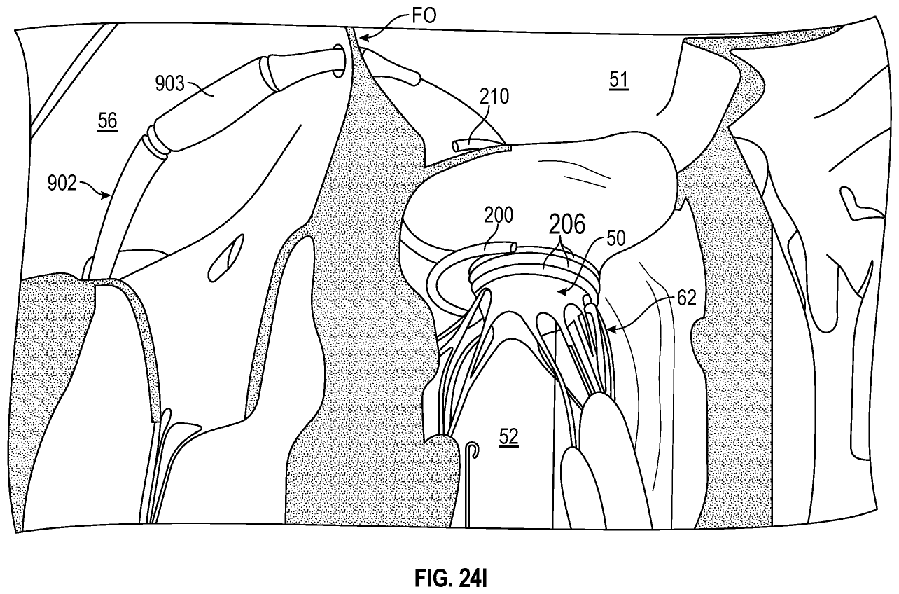

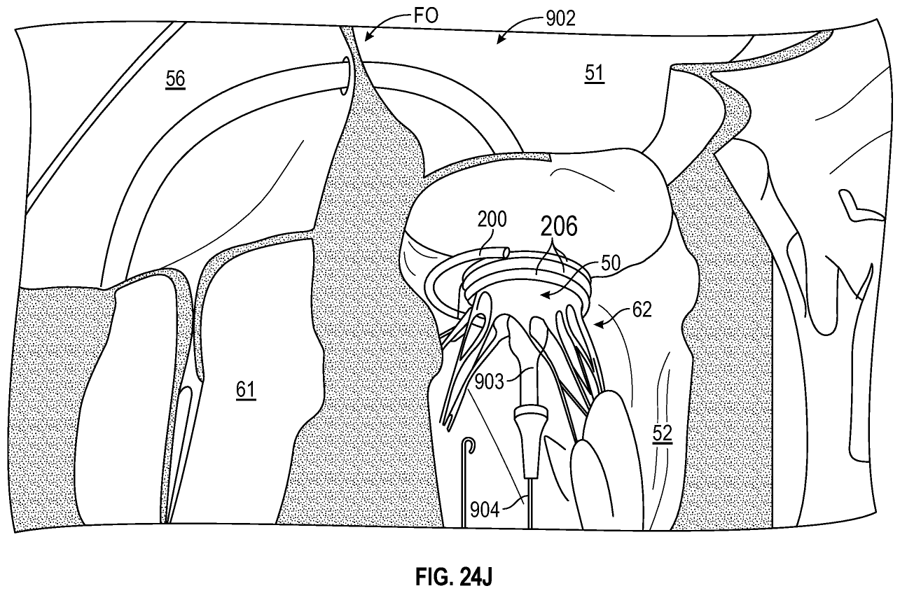

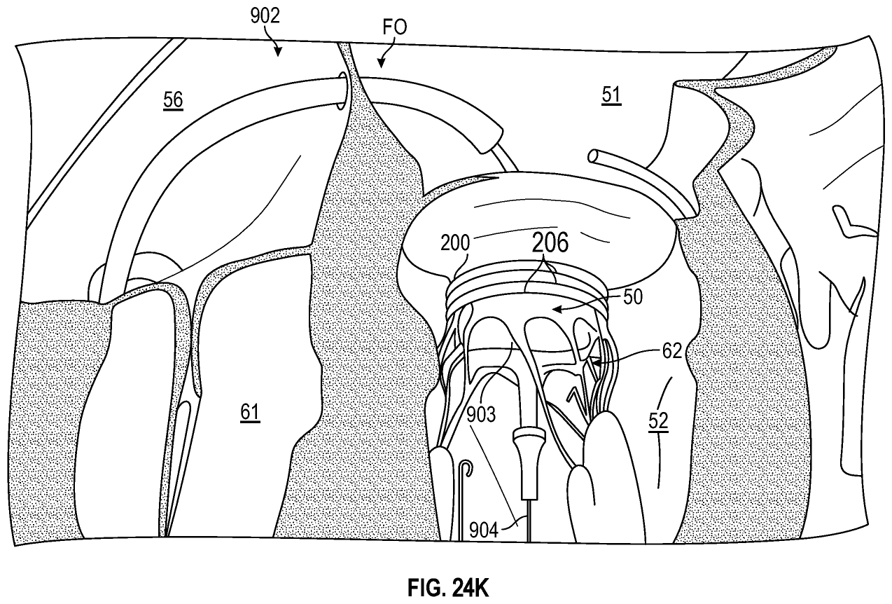

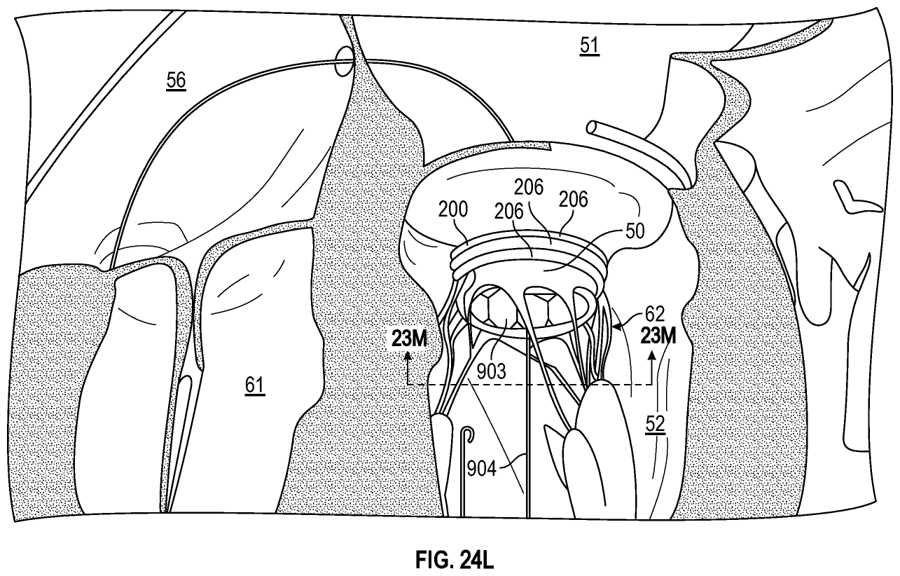

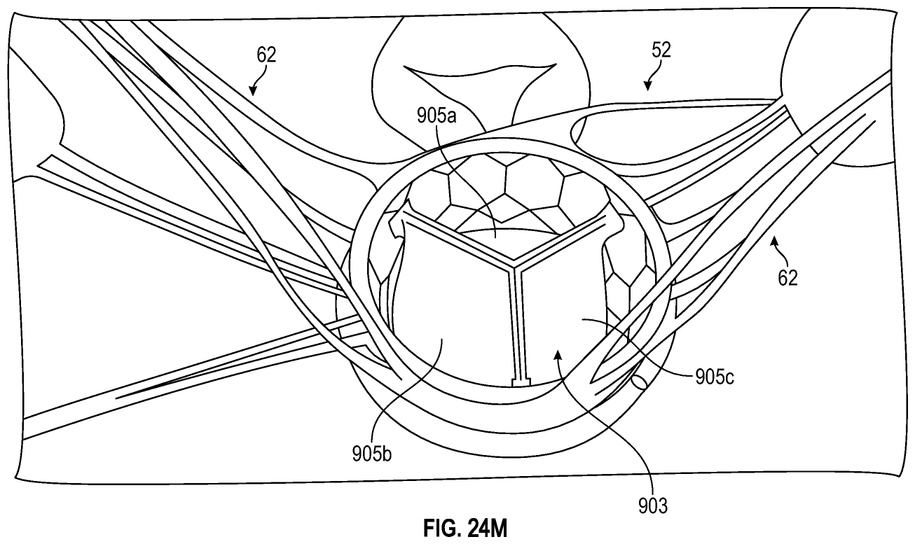

The docking coil 200 may be configured to be deployed to the mitral valve by a docking coil sleeve 212, as shown in FIG. 14A extending over the docking coil 200. The docking coil 200 may be positioned within a lumen of the docking coil sleeve 212. The docking coil 200 may be deployed by being wrapped around the leaflets of the mitral valve and other native structure, including chordae, within the docking coil sleeve 212. The turns of the docking coil 200 wrapping around the structure of the mitral valve are shown in FIGS. 24A-24C, for example, and the stabilization turn 210 being deployed within the atrium is shown in FIGS. 24D-24H for example.

The docking coil 200 may be configured to have an outer surface that is configured to produce a frictional securement to the structure of the mitral valve. As such, the outer surface upon contact with the structure of the mitral valve is configured to provide friction to secure the docking coil 200 in position.

The docking coil sleeve 212 is configured to extend over the docking coil 200 to reduce friction between the docking coil 200 and the structure of the mitral valve during deployment, by being positioned between the docking coil 200 and the structure of the mitral valve, such as the mitral valve leaflets. With the docking coil 200 and docking coil sleeve 212 in position, the docking coil sleeve 212 may be retracted relative to the docking coil 200 to leave the docking coil 200 in position upon the mitral valve leaflets.

FIG. 14A illustrates a side view of an example of a docking coil sleeve 212 that may be utilized according to examples herein. The docking coil sleeve 212 may include a distal tip 214 and a proximal end 216 and a length extending from the distal tip 214 to the proximal end 216. The docking coil sleeve 212 may include an outer surface 218 that may be configured to be lubricious to reduce friction between the docking coil sleeve 212 and the structure of the mitral valve as the sleeve extends around the structure of the mitral valve during deployment.

FIG. 14B illustrates a partial cross sectional view of the docking coil sleeve 212 shown in FIG. 14A. The docking coil sleeve 212 may include an interior lumen 220 that may be configured for the docking coil 200 to slide within. The interior lumen 220 may face opposite the lubricious outer surface 218. The interior lumen 220 may extend distally to the distal tip 214. A wall 222 of the docking coil sleeve 212 may extend around the interior lumen 220.

The distal tip 214 may have an aperture for the docking coil 200 to pass through to deploy from the docking coil sleeve 212.

The docking coil sleeve 212 may be configured to be flexible in examples, to contour around the native mitral leaflets with the docking coil 200 positioned within the interior lumen 220. The docking coil sleeve 212 accordingly may form a coil when extending around the native mitral leaflets, to account for the coil shape of the docking coil 200 positioned within the interior lumen 220.

An issue may arise when the leading turn 202 of the docking coil 200 within the docking coil sleeve 212 is being wrapped around the native mitral valve leaflets. A potential complication is that if the diameter of the leading turn 202 shown in FIG. 13A is too large, then the leading tip 204 of the docking coil 200 or the distal tip 214 of the docking coil sleeve 212 may undesirably contact a surface within the patient's heart, which may comprise a wall within the left ventricle or other structure such as chordae. As such, it may be desirable to utilize a docking coil sleeve 212 that may be deflectable to allow for navigation of the docking coil sleeve 212 around the mitral valve leaflets.

In examples herein, the docking coil sleeve 212 may include a tether 224 that may extend along at least a portion of the docking coil sleeve 212 and may be configured to deflect the docking coil sleeve 212. The tether 224 may be configured to deflect the distal tip 214 of the docking coil sleeve 212.

The tether 224 may extend along a tether lumen 226 that may extend along all or a portion of the docking coil sleeve 212. The tether 224 may be configured to be positioned at a distal portion 228 of the docking coil sleeve 212 and may be configured to be positioned along an inner curve of the distal portion 228 of the docking coil sleeve 212 as the docking coil sleeve 212 wraps around the native structure of the mitral valve. In examples, the tether 224 may be positioned at other locations as desired. The tether 224 may have a distal end that may couple to a securing device such as a securing ring 230 that may be positioned at the distal tip 214 of the docking coil sleeve 212 or at another position as desired.

The tether 224 may be configured to be retracted proximally to deflect the docking coil sleeve 212 in a direction towards the tether 224. The tether 224 may comprise a pull tether in examples as desired.

In examples, the tether 224 may include a proximal portion 229 that may extend exterior of the docking coil sleeve 212 for engagement and retraction during use.

FIG. 14C illustrates a cross sectional view of the docking coil sleeve 212 along line 14C-14C in FIG. 14B.

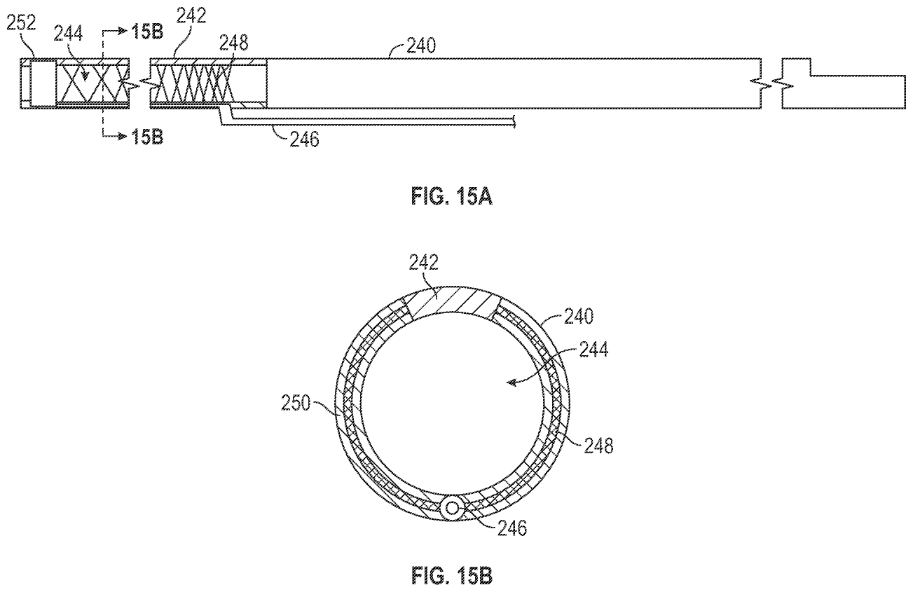

Variations in the configuration of the docking coil sleeve 212 may be provided. FIGS. 15A and 15B for example, illustrate an example in which a docking coil sleeve 240 includes a spine 242 extending along at least a portion of the docking coil sleeve 240. The spine 242 may be positioned opposed circumferentially to the tether 246. The spine 242 may be configured to oppose a deflection of the docking coil sleeve 240 in a direction towards the tether 246. As such, the spine 242 may provide a resilient force that deflects the docking coil sleeve 240 in an opposite direction upon release of the tether 246.

The docking coil sleeve 240 may further include a braid 248 that may be positioned within the wall 250. The braid 248 accordingly may comprise a braid layer. The braid 248 may extend around the interior lumen 244. The braid 248 in examples may be positioned at a distal end portion of the docking coil sleeve 240.

As shown in FIG. 15A, the braid 248 in examples may have a looser braid configuration in a distal portion of the braid 248 relative to a proximal portion of the braid 248. As such, the braid 248 may be configured to have a greater deflection closer to the distal end 252 of the docking coil sleeve 240 than a proximal portion of the docking coil sleeve 240. The braid 248 may have a flexibility that increases in a direction towards a distal tip of the docking coil sleeve 240. The docking coil sleeve 240 may accordingly have a greater deflection at the distal end 252 than a proximal portion of the docking coil sleeve 240 upon a deflection force being applied by the tether 246. FIG. 15B illustrates a cross sectional view along line 15B-15B in FIG. 15A.

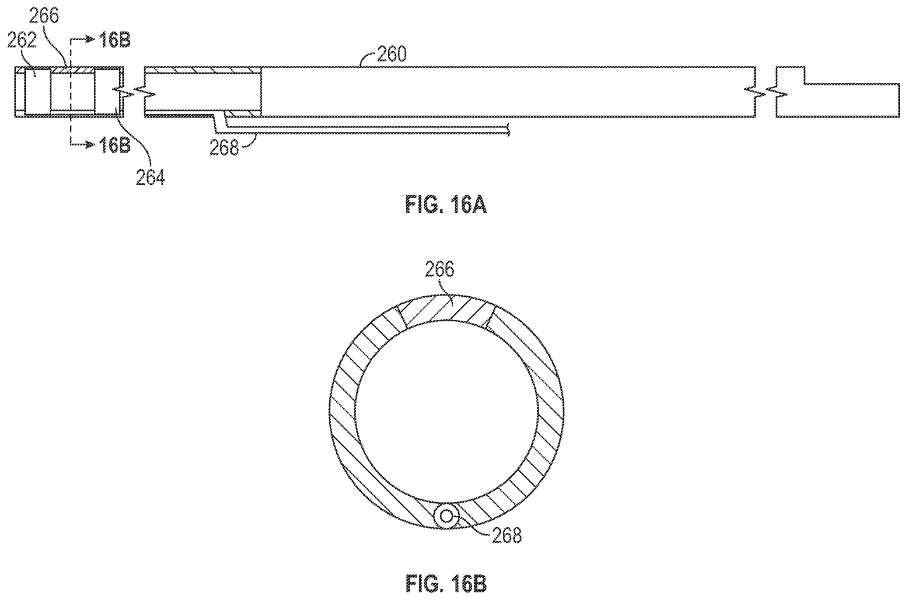

FIGS. 16A and 16B illustrate an example of a docking coil sleeve 260 including a first retainer ring 262 and a second retainer ring 264 positioned at a spaced relationship from each other. A spine 266 may extend between the first retainer ring 262 and the second retainer ring 264, and may operate in a similar manner as the spine 242 shown in FIGS. 15A and 15B. The space between the retainer rings 262, 264 may define a region for the docking coil sleeve 260 to deflect due to retraction of the tether 268. FIG. 16B illustrates a cross sectional view along line 16B-16B in FIG. 16A.

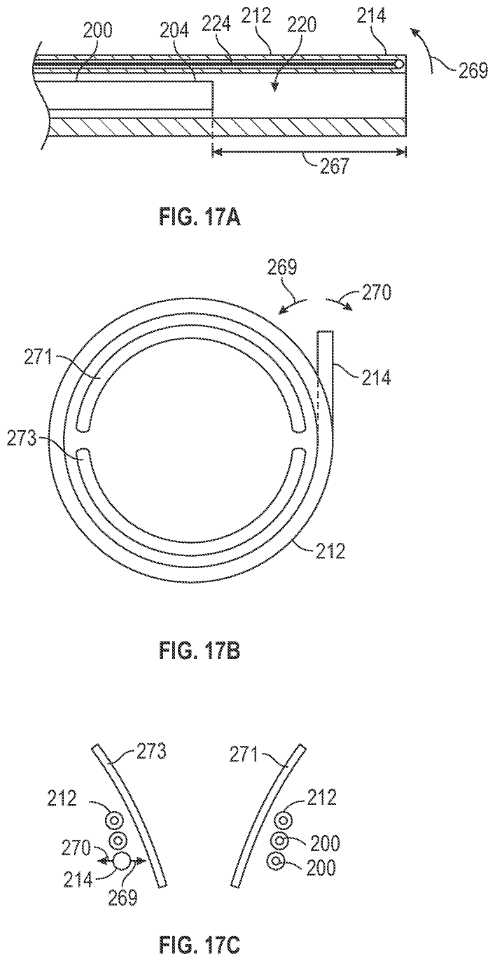

FIGS. 17A-17C illustrate an exemplary operation of a docking coil sleeve that includes a tether for deflection as disclosed in examples herein. FIG. 17A, for example, illustrates a docking coil 200 within the interior lumen 220 of the docking coil sleeve 212 shown in FIG. 14B for example. The distal or leading tip 204 of the docking coil 200 may be positioned at a distance 267 from the distal tip 214 of the docking coil sleeve 212. The distal tip 214 of the docking coil sleeve 212 accordingly may overhang the distal tip 204 of the docking coil 200. The space within the lumen 220 between the distal or leading tip 204 of the docking coil 200 and the distal tip 214 of the docking coil sleeve 212 may enhance the flexibility of the distal tip 214 of the docking coil sleeve 212 upon a longitudinal force being applied to the tether 224.

The longitudinal force applied to the tether 224 may deflect the distal tip 214 in the direction of the arrow 269 shown in FIG. 17A. In a configuration in which the docking coil sleeve 212 forms a coil, the direction of deflection indicated by arrow 269 may be radially inwards.

FIG. 17B illustrates a top schematic view of the operation of the docking coil sleeve 212 extending around leaflets 271, 273 of a mitral valve, with the upper turns of the docking coil sleeve 212 visible and the distal tip 214 of the docking coil sleeve 212 shown comprising a leading portion of the docking coil sleeve 212. The docking coil sleeve 212 may be deflectable via operation of the tether 224, and may be deflectable radially inward as represented by the arrow 269 shown in FIG. 17B. Further, the distal tip 214 may be deflectable radially outward via the release of the tether 224. The arrow 270 may represent the deflection due to the release of the tether 224. In examples, such as shown in FIGS. 15A-16B, a spine 242, 266 may cause the distal tip 214 to deflect radially outward upon release of the tether 224. In examples, such as shown in FIGS. 15A-15B, a braid 248 may be utilized to locate the deflection at the distal tip 214. In examples, such as shown in FIGS. 16A-16B, a deflectable portion between retainer rings 262, 264 may be utilized to locate the deflection at the distal tip 214. Various combinations of features may be utilized in examples.

FIG. 17C illustrates a side view of the docking coil sleeve 212 extending around the mitral valve leaflets 271, 273, with the distal tip 214 being deflectable due to operation of the tether 224. The docking coil sleeve 212 may be deflectable radially inward or outward utilizing the tether 224. The docking coil sleeve 212 may be deflected with the tether 224. The tether 224 may be retracted to deflect the docking coil sleeve 212.

The turns of the docking coil 200 within the docking coil sleeve 212 may extend in a ventricular direction as shown in FIG. 17C. In examples, another form of encircling may be utilized.

The docking coil 200 within the docking coil sleeve 212 may encircle the mitral valve leaflets upon being deployed from the delivery catheter, as may be disclosed herein. The docking coil sleeve 212 may be deflected with the tether 224 during the encircling of the mitral valve leaflets.

With the docking coil sleeve 212 and the docking coil 200 in the desired position, the docking coil 200 may be deployed from the docking coil sleeve 212 to the implantation site by the docking coil sleeve 212 being retracted proximally relative to the docking coil 200. The docking coil 200 may accordingly remain in position upon the mitral valve leaflets.

In examples, a deflection mechanism similar to the deflection mechanism 112 shown in FIG. 4 may engage a proximal portion of the tether 224 to allow the tether 224 to be retracted to deflect the docking coil sleeve 212. In examples, other forms of deflection mechanisms may be utilized as desired.

A deflectable distal tip of the docking coil sleeve may beneficially allow for reduced possibility of undesired contact with the structure of the native heart valve, which may include a ventricular wall or undesired contact with chordae. Further, a deflectable distal tip of the docking coil sleeve may allow for enhanced control of the docking coil sleeve to encircle desired native structures such as mitral valve leaflets and chordae.

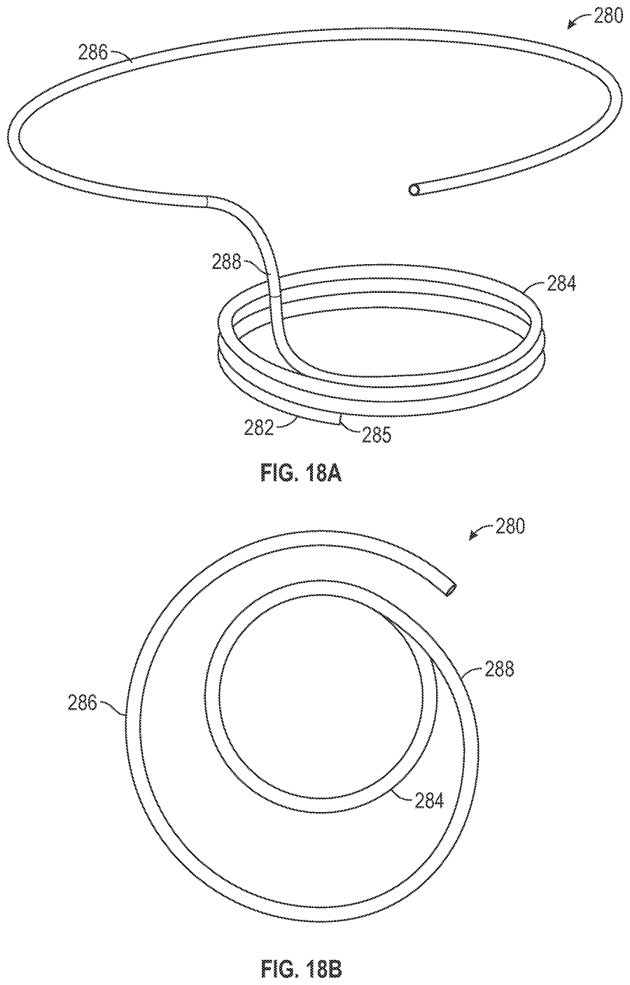

FIGS. 18A-22C illustrate examples in which a leading portion of a docking coil sleeve may have an orientation that is different than an orientation of a leading portion of a docking coil. A leading tip of the docking coil sleeve may be configured to slide relative to the leading tip of the docking coil to deflect the leading tip of the docking coil or the leading tip of the docking coil sleeve radially inward or outward.

FIG. 18A illustrates an example of a docking coil 280 that may be utilized according to examples herein. The docking coil 280 may include a leading portion 282 in the form of a leading turn that may have a lesser diameter than the leading turn 202 shown in FIG. 13A. For example, the leading portion 282 may have a diameter that matches a diameter of the functional turns 284, and thus may have a lesser radius of curvature than the leading turn 202 shown in FIG. 13A.

The leading portion 282 may have an orientation (e.g., a curved orientation as shown in FIG. 18A) and may extend to a leading tip 285 of the docking coil 280.

A configuration of a stabilizing turn 286 and a transition curve 288 may be similar to the respective configurations of the stabilizing turn 210 and the transition curve 208 shown in FIG. 13A.

FIG. 18B illustrates a top view of the docking coil 280 shown in FIG. 18A.

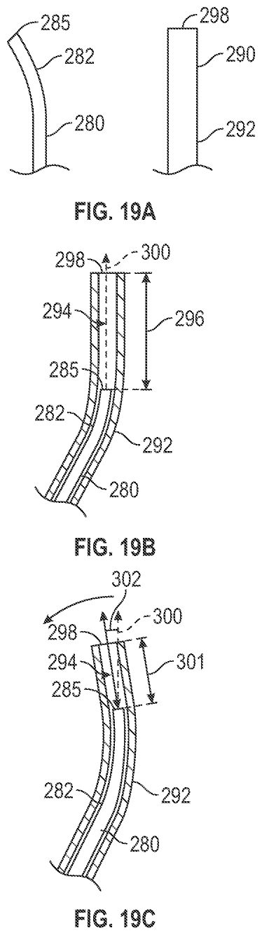

FIG. 19A illustrates a close up view of the leading portion 282 of the docking coil 280 relative to a leading portion 290 of a docking coil sleeve 292. As shown in FIG. 19A, the leading portion 282 of the docking coil 280 may have an orientation that is curved with a defined radius of curvature.

A leading portion 290 of a docking coil sleeve 292 may extend to a leading tip 298 of the docking coil sleeve 292. The leading portion 290 may have an orientation that is different than the orientation of the leading portion 282 of the docking coil 280. As shown in FIG. 19A, for example, the leading portion 290 of the docking coil sleeve 292 may have a straightened configuration. In other examples, other orientations may be utilized, including curved orientations having a different radius of curvature of the docking coil 280, among other orientations.

Referring to FIG. 19B, the docking coil 280 may be positioned within an interior lumen 294 of the docking coil sleeve 292. The interior lumen 294 of the docking coil sleeve 292 may be configured for the docking coil 280 to slide within. The leading tip 285 of the docking coil 280 may be positioned at a distance 296 from the leading tip 298 of the docking coil sleeve 292. The leading tip 298 of the docking coil sleeve 292 extend in a direction marked by line 300 in FIG. 19B.

The docking coil 280 may be slidable within the interior lumen 294 of the docking coil sleeve 292 and may be slidable distally and proximally within the docking coil sleeve 292. The docking coil 280 sliding within the interior lumen 294 of the docking coil sleeve 292 may vary the distance 296 of the leading tip 285 of the docking coil 280 from the leading tip 298 of the docking coil sleeve 292.

The variation in the distance 296 of the leading tip 285 of the docking coil 280 from the leading tip 298 of the docking coil sleeve 292 may deflect the leading tip 298 of the docking coil sleeve 292. For example, as shown in FIG. 19C, upon the docking coil 280 being advanced distally relative to the leading tip 298 of the docking coil sleeve 292, the distance 301 between the leading tip 285 of the docking coil 280 and the leading tip 298 of the docking coil sleeve 292 decreases from the distance 296 shown in FIG. 19B.

Due to the radius of curvature of the leading portion 282 of the docking coil 280, the docking coil sleeve 292 may accordingly conform to the curvature of the leading portion 282 and deflect according to the curvature of the leading portion 282. FIG. 19C, for example, illustrates a variation in the angle of deflection 302 of the leading tip 298 of the docking coil sleeve 292 from the direction represented by the line 300 shown in FIG. 19B. As such, the leading tip 298 of the docking coil sleeve 292 may deflect from the position shown in FIG. 19B due to the sliding movement of the docking coil 280 within the interior lumen 294.

The docking coil 280 may be retracted to allow the docking coil sleeve 292 to return to the configuration shown in FIG. 19B. For example, the docking coil sleeve 292 may be biased to return to the configuration shown in FIG. 19B upon retraction of the docking coil 280.

The relative position of the leading tip 298 of the docking coil sleeve 292 and the leading tip 285 of the docking coil 280 may be varied to allow the leading tip 298 of the docking coil sleeve 292 to deflect during deployment of the docking coil 280. For example, the distance between the tips 285, 298 may be varied to cause a deflection during the encircling of the mitral valve leaflets.

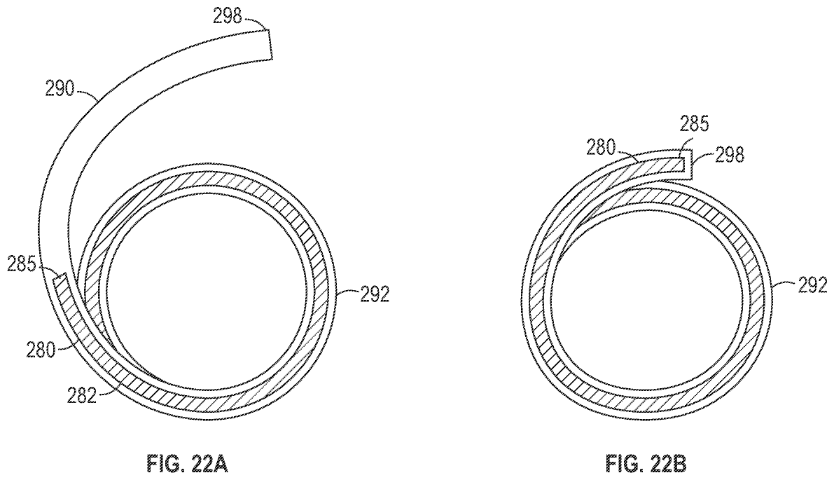

FIGS. 22A and 22B, for example, illustrate such an operation. The docking coil 280 is shown extending within the docking coil sleeve 292, with the leading tip 285 of the docking coil 280 at a distance from the leading tip 298 of the docking coil sleeve 292 in FIG. 22A. The leading portion 290 of the docking coil sleeve 292 may have a preset radius of curvature, which may be larger than a preset radius of curvature of the leading portion 282 of the docking coil 280. Further, the orientation of the leading portion 282 of the docking coil 280 may be configured to form a diameter that is less than a diameter of the leading portion 290 of the docking coil sleeve 292 as shown in FIG. 22A.

Sliding the leading tip 285 of the docking coil 280 distally relative to the leading tip 298 of the docking coil sleeve 292 may deflect the leading tip 298 of the docking coil sleeve 292 radially inward as shown in FIG. 22B for example. Further, sliding the leading tip 285 of the docking coil 280 proximally relative to the leading tip 298 of the docking coil sleeve 292 may deflect the leading tip 298 of the docking coil sleeve 292 radially outward. Such an operation would return the docking coil sleeve 292 to the position shown in FIG. 22A for example. Upon the docking coil 280 being in the desired position, the docking coil sleeve 292 may be fully retracted to leave the docking coil 280 in place upon the mitral valve leaflets.

In the example shown in FIG. 19A, the docking coil sleeve 292 may have a straightened configuration. In examples, the docking coil sleeve 292 may have a preset curvature that may yet be deflected by a different curvature of the docking coil 280.

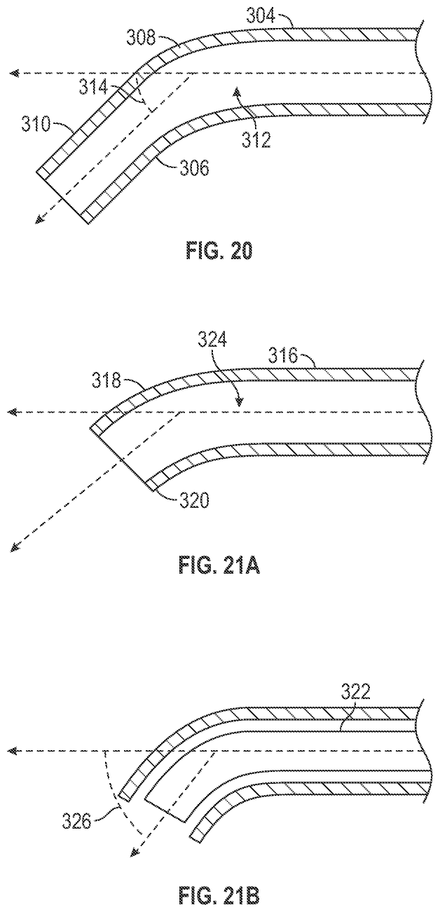

FIG. 20, for example, illustrates an example of a docking coil sleeve 304 that has a leading portion 306 with a preset curvature. The leading portion 306 may include a curved portion 308 and a straightened portion 310 distal of the curved portion 308. A docking coil may be passed through the interior lumen 312 to deflect the leading tip and vary an angle of deflection 314 of the leading tip.

FIG. 21A illustrates an example of a docking coil sleeve 316 including a leading portion 318 that has a preset curvature. The leading tip 320 of the docking coil sleeve 316 retains the curvature of the leading portion 318. FIG. 21B illustrates a docking coil 322 having been passed through the interior lumen 324 of the docking coil sleeve 316 to deflect the leading tip 320 and vary the angle of deflection 326 of the leading tip 320.

The deflection of the docking coil sleeves may allow the docking coil sleeves to be deflected during deployment of the docking coils. Such deflection may avoid undesirable contact with native structures and may aid in encircling structures such as the mitral valve leaflets and chordae. The deflection may thus produce a similar result as the deflection represented in FIG. 17C with arrows 269 and 270.

Upon the docking coil sleeve 292 and the docking coil 280 being placed in the desired position, for example around leaflets of the mitral valve, the docking coil sleeve 292 may be retracted relative to the docking coil 280. The docking coil 280 may remain in position to be deployed to the mitral valve leaflets with the docking coil sleeve 292 being removed from the patient's ventricle.

The relative positions of the docking coil sleeve 292 and the docking coil 280 may be controlled with a control mechanism that may control the relative position of the leading tips 298, 285 and the variation in the distance between the leading tips 298, 285. For example, a control mechanism may couple to proximal portions of the docking coil sleeve 292 and the docking coil 280 to control and vary the distance between the leading tips 298, 285.

In examples, the docking coil sleeve 292 may include a spine as shown in FIGS. 15A and 15B, or a braid as shown in FIGS. 15A and 15B, or a spine extending between retainer rings as shown in FIGS. 16A and 16B. The spine, for example, may extend along a leading portion of the docking coil sleeve. The braid may be positioned at the leading portion of the docking coil sleeve. Such features may bias the docking coil sleeve 292 back to a preset orientation of the docking coil sleeve 292 upon the docking coil 280 being retracted proximally. Various combinations of features may be provided as desired.

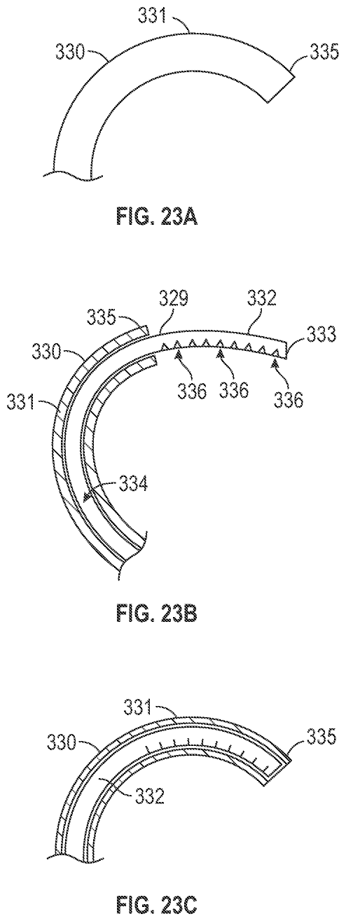

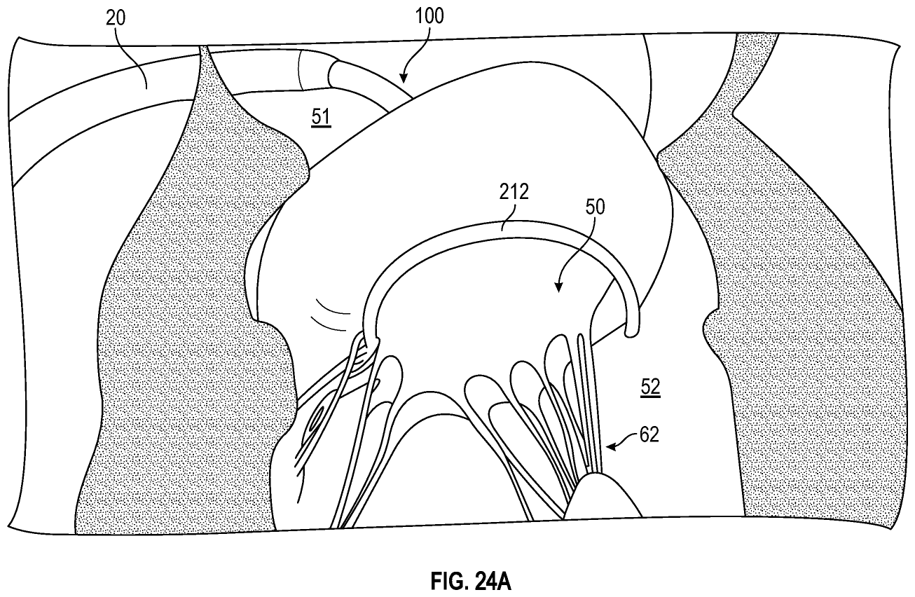

In examples, a docking coil may comprise the leading portion of the combination of the docking coil and docking coil sleeve that encircles the mitral valve leaflets. FIGS. 23A-23C for example, illustrate such an example, in which a docking coil sleeve 330 may have a preset curvature as shown in FIG. 23A for example. A docking coil 332 may be positioned within an interior lumen 334 of the docking coil sleeve 330 as shown in FIG. 23B for example. The docking coil 332 may include one or more cuts 336 upon the docking coil 332 that may allow the leading tip 333 of the docking coil 332 to deflect upon the docking coil sleeve 330 extending over the docking coil 332. The one or more cuts 336 may be positioned on an inner curve of the docking coil 332.

The orientation of the leading portion 329 of the docking coil 332 may be configured to form a diameter that is greater than a diameter of the leading portion 331 of the docking coil sleeve 330. The leading portion 331 of the docking coil sleeve 330 may have a preset radius of curvature that is smaller than a preset radius of curvature of the leading portion 329 of the docking coil 332. As such, sliding the leading tip 335 of the docking coil sleeve 330 distally relative to the leading tip 333 of the docking coil 332 may deflect the leading tip 333 of the docking coil 332 radially inward.

FIG. 23C, for example, illustrates the docking coil sleeve 330 having been advanced distally to cause the docking coil 332 to deflect and vary an angle of deflection of the docking coil 332.

The examples of FIGS. 18A-23C may allow the docking coil sleeve and/or docking coil to deflect during deployment and thus avoid undesired contact with native structure or to better encircle native structure such as mitral leaflets or chordae.

FIGS. 24A-24M illustrate steps involving further deployment of the anchoring device in the form of the docking coil and further implantation of a prosthetic implant to the anchoring device. The steps may continue from the catheter device being in the position shown in FIG. 12B.

FIG. 24A illustrates the delivery catheter 100 deploying a docking coil sleeve 212 through the commissure A3P3 and around the chordae tendineae 62 and native leaflets in the left ventricle 52 of the patient's heart. The anchoring device or a leading portion or encircling coil/turn of the anchoring device may exit the distal aperture of the delivery catheter 100 and may begin to take a shape set or shape memory form in the direction of the delivery catheter 100. The anchoring device may be positioned within the docking coil sleeve 212. The anchoring device may comprise a docking coil that is passed through the interior lumen of the catheter 100.

Referring to FIG. 24B, the docking coil sleeve 212 can be further deployed from the delivery catheter 100, such that the docking coil sleeve 212 wraps around the chordae tendineae 62 in a position that is substantially parallel to the plane of the mitral valve 50. The docking coil sleeve 212 may be deflected according to examples herein during the encircling procedure.

Referring to FIG. 24C, the docking coil sleeve 212 is disposed around the chordae tendineae 62 to loosely position the anchoring device on the ventricular side of the mitral valve for holding a heart valve. In the illustrated example, the docking coil sleeve 212 is disposed in the left ventricle 52 such that functional coils 340 of the anchoring device and the docking coil sleeve 212 are wrapped closely around the chordae tendineae and/or native leaflets. The lower end turn/coil or encircling turn/coil in examples may extend outwardly somewhat because of its larger radius of curvature. In some examples, the anchoring device can include less than three coils or more than three coils that are disposed around the chordae tendineae and/or leaflets.

Upon the anchoring device being in the desired position, the docking coil sleeve may be retracted to leave the anchoring device in position upon the mitral valve leaflets.

FIG. 24D illustrates the delivery catheter 100 in the left atrium 51 in a position after the coils of the anchoring device are disposed around the chordae tendineae 62 and native leaflets (as shown in FIG. 24C). In this position, the distal tip 106 of the delivery catheter 100 is substantially parallel with the plane of the mitral valve 50 and is located at or near (e.g., extending slightly into or through, such as 1-5 mm or less) the commissure A3P3 of the mitral valve 50.

Referring to FIG. 24E, the delivery catheter may be translated or retracted axially along the anchoring device in the direction X and into the outer sheath 20. Translation or retracting of the delivery catheter can cause the portions of the anchoring device positioned one the atrial side of the native valve (e.g., in the atrium) to be unsheathed and released from the delivery catheter. For example, this can unsheath and release any upper portion of any functional coil and/or upper coil positioned on the atrial side of the native valve (if any). In one exemplary example, the anchoring device does not move or does not substantially move as the delivery catheter is translated, e.g., a pusher can be used to hold the anchoring device in place and/or inhibit or prevent retraction of the anchoring device when the delivery catheter is retracted.

Examples of pushers that may be utilized in examples herein may be disclosed in International Patent Application PCT/US2020/036577, filed Jun. 8, 2020, and titled “Systems, Devices, and Methods for Treating Heart Valves,” and published as WO/2020/247907, which is incorporated by reference herein in its entirety.

Referring to FIG. 24F, in the illustrated example, translation or retraction of the delivery catheter can also unsheath/release any upper end coil/turn (e.g., a larger diameter stabilization coil/turn) of the anchoring device or docking coil 200 from the delivery catheter. As a result of the unsheathing/releasing, the atrial side of the anchoring device or upper coil (e.g., stabilization coil with a larger diameter or radius of curvature) extends out of the delivery catheter 100 and begins to assume its preset or relaxed shape-set/shape-memory shape. In examples, the anchoring device can also include an upward extending portion or connecting portion that extends upward from a bend Z and can extend and/or bridge between an upper end stabilization coil/turn and other coil/turns of the anchoring device (e.g., functional coils/turns). In some examples, the anchoring device can have only one upper coil on the atrial side of the native valve. In some examples, the anchoring device can include more than one upper coil on the atrial side of the native valve.

Referring to FIG. 24G, the delivery catheter 100 continues to translate back into the outer sheath or guide sheath 20, which causes the upper portion of the anchoring device to be released from inside the delivery catheter. The anchoring device is connected closely to the pusher 950 by an attachment means, such as suture/line 901 (other attachment or connection means can also be used as desired). The upper end coil/turn or stabilization coil/turn is shown as being disposed along the atrial wall to temporarily and/or loosely hold the position or height of the anchoring device relative to the mitral valve 50.

Referring to FIG. 24H, the anchoring device is fully removed from a lumen of the delivery catheter 100, and slack is shown in a suture/line 901 that is removably attached to the anchoring device, e.g., suture/line 901 can loop through an eyelet at the end of the anchoring device. To remove the anchoring device from the delivery catheter 100, the suture 901 is removed from the anchoring device. However, before the suture 901 is removed, the position of the anchoring device can be checked. If the position of the anchoring device or docking coil 200 is incorrect, the anchoring device can be pulled back into the delivery catheter by the pusher 950 (e.g., a pusher rod, pusher wire, pusher tube, etc.) and redeployed.

Referring to FIG. 24I, after the delivery catheter 100 and the outer sheath 20 are detached from the anchoring device, a heart valve delivery device/catheter 902 can be used to deliver a heart valve 903 to the mitral valve 50. The heart valve delivery device 902 may utilize one or more of the components of the delivery catheter 100 and/or outer or guide sheath 20 or the delivery device 902 may be independent of the delivery catheter 100 and outer or guide sheath. In the illustrated example, the heart valve delivery device 902 enters the left atrium 51 using a transseptal approach. In examples, the heart valve delivery catheter 902 may be passed through the outer sheath 20. The heart valve delivery catheter 902 may deploy the prosthetic heart valve to dock with an anchoring device in the form of a docking coil.

Examples of implants that may be utilized in examples herein for docking with the anchoring device may be disclosed in International Patent Application PCT/US2020/036577, filed Jun. 8, 2020, and titled “Systems, Devices, and Methods for Treating Heart Valves,” and published as WO/2020/247907, which is incorporated by reference herein in its entirety.

Referring to FIG. 24J, the heart valve delivery device/catheter 902 is moved through the mitral valve 50 such that heart valve 903 is placed between the leaflets of the mitral valve and the anchoring device. The heart valve 903 can be guided along a guide wire 904 to the deployment position.

Referring to FIG. 24K, after the heart valve 903 is placed in the desired position, an optional balloon is expanded to expand the heart valve 903 to its expanded, deployed size. That is, the optional balloon is inflated such that the heart valve 903 engages the leaflets of the mitral valve 50 and forces the ventricular turns outward to an increased size to secure the leaflets between the heart valve 903 and the anchoring device. The outward force of the heart valve 903 and the inward force of the coil can pinch the native tissue and retain the heart valve 903 and the coil to the leaflets. In some examples, a self-expanding heart valve can be retained in a radially compressed state within a sheath of the heart valve delivery device 902, and the heart valve can be deployed from the sheath, which causes the heart valve to expand to its expanded state. In some examples, a mechanically expandable heart valve is used or a partially mechanically expandable heart valve is used (e.g., a valve that may expand by a combination of self-expansion and mechanical expansion).

Referring to FIG. 24L, after the heart valve 903 is moved to its expanded state, the heart valve delivery device 902 and the wire 904 are removed from the patient's heart. Further, the guide sheath 20 may be removed from the patient's heart as well. The heart valve 903 is in a functional state and replaces the function of the mitral valve 50 of the patient's heart.

FIG. 24M shows the heart valve 903 from an upward view in the left ventricle 52. In FIG. 24M, the heart valve 903 is in the expanded and functional state. In the illustrated example, the heart valve 903 includes three valve members 905a-c (e.g., leaflets) that are configured to move between an open position and a closed position. In alternative examples, the heart valve 903 can have more than three valve members or less than three valve members that are configured to move between an open position and a closed position, such as, for example, two or more valve members, three or more valve members, four or more valve members, etc. In the illustrated example, the valve members 905a-c are shown in the closed position, which is the position the valve members are in during the systolic phase to prevent blood from moving from the left ventricle and into the left atrium. During the diastolic phase, the valve members 905a-c move to an open position, which allows blood to enter the left ventricle from the left atrium.

While the examples illustrated herein show the delivery catheter 100 delivering an anchoring device in the form of a docking coil 200 through the commissure A3P3, it should be understood that the delivery catheter 100 can take a configuration and be positioned to deliver the anchoring device through the commissure A1P1, such that the anchoring device can be wrapped around the chordae tendineae in the left ventricle of the patient's heart. In addition, while the illustrated examples show the delivery catheter 100 delivering an anchoring member to the mitral valve and the heart valve delivery device 902 delivering a heart valve 903 to the mitral valve 50, it should be understood that the anchoring device and the heart valve 903 can be used mutatis mutandis to repair the tricuspid valve, the aortic valve, or the pulmonary valve.

Examples as disclosed herein may be utilized in such a method. For example, any example of delivery catheter, docking coil, or docking coil sleeve disclosed herein may be utilized as desired. In examples, the components may be utilized separately as desired.

The delivery catheter configurations described herein provide examples that allow for accurate positioning and deployment of an anchoring device. However, in some instances, retrieval or partial retrieval of the anchoring device can still be necessary at any stage during or after deployment of the anchoring device in order, for example, to reposition the anchoring device at the native valve, or to remove the anchoring device from the implant site. Various locks or lock-release mechanisms may be used for attaching and/or detaching an anchoring or docking device from a deployment pusher that pushes the anchoring device out of the delivery catheter. Other locks or locking mechanisms are also possible, e.g., as described in U.S. Provisional Patent Application Ser. No. 62/560,962, filed on Sep. 20, 2017 incorporated by reference herein. The anchoring device can be connected at its proximal side to a pusher or other mechanism that can push, pull, and easily detach from the anchoring device. Further features of the systems, apparatuses, and methods disclosed herein that may be utilized are described in U.S. patent application Ser. No. 15/984,661 (U.S. Publication No. 2018/0318079), filed May 21, 2018, the entire contents of which are incorporated by reference herein.

In examples, the various manipulations and controls of the systems and devices described herein can be automated and/or motorized. For example, the controls or knobs described above can be buttons or electrical inputs that cause the actions described with respect to the controls/knobs above. This can be done by connecting (directly or indirectly) some or all of the moving parts to a motor (e.g., an electrical motor, pneumatic motor, hydraulic motor, etc.) that is actuated by the buttons or electrical inputs. For example, the motor can be configured, when actuated, to cause tethers such as control wires or pull wires to tension or relax to move the distal region of the catheter. Additionally or alternatively, the motor could configured, when actuated, to cause a device such as a pusher to move translationally or axially relative to the catheter to cause an anchoring or docking device to move within and/or into or out of the catheter. Automatic stops or preventative measures could be built in to prevent damage to the system/device and/or patient, e.g., to prevent movement of a component beyond a certain point.

It should be noted that the devices and apparatuses described herein can be used with other surgical procedures and access points (e.g., transapical, open heart, etc.). It should also be noted that the devices described herein (e.g., the deployment tools) can also be used in combination with various other types of anchoring devices and/or prosthetic valves different from the examples described herein.

For purposes of this description, certain aspects, advantages, and novel features of the examples of this disclosure are described herein. The disclosed methods, apparatuses, and systems should not be construed as limiting in anyway. Instead, the present disclosure is directed toward all novel and nonobvious features and aspects of the various disclosed examples, alone and in various combinations and sub-combinations with one another. The methods, apparatuses, and systems are not limited to any specific aspect or feature or combination thereof, nor do the disclosed examples require that any one or more specific advantages be present or problems be solved. Features, elements, or components of one example can be combined into other examples herein.

Example 1: A system for delivering an implant to a portion of a patient's body. The system may include a delivery catheter including an elongate shaft having an interior lumen for the implant to pass through and a distal end portion including a first flexible portion and a second flexible portion that is positioned distal of the first flexible portion, the first flexible portion including a first tether and a first linear spine that is positioned opposed circumferentially to the first tether, and the first flexible portion is configured to deflect in a plane upon a longitudinal force being applied to the first tether, and the second flexible portion including a second tether and a second linear spine that is positioned non-orthogonal and non-parallel relative to the first linear spine, and the second flexible portion is configured to deflect in a direction that is non-orthogonal and non-parallel with the plane upon a longitudinal force being applied to the second tether.

Example 2: The system of any example herein, in particular Example 1, wherein the second tether is positioned opposed circumferentially to the second linear spine.

Example 3: The system of any example herein, in particular Example 1 or Example 2, wherein the second tether is positioned at an obtuse angle relative to the first tether.

Example 4: The system of any example herein, in particular Example 1, wherein the second tether is positioned orthogonal relative to the first tether.

Example 5: The system of any example herein, in particular Examples 1-4, wherein the second linear spine is positioned at an obtuse angle relative to the first linear spine.

Example 6: The system of any example herein, in particular Examples 1-5, wherein the second linear spine is positioned at an acute angle relative to the first tether.

Example 7: The system of any example herein, in particular Examples 1-6, wherein the direction that the second flexible portion is configured to deflect in is obtuse relative to a direction of deflection of the first flexible portion.

Example 8: The system of any example herein, in particular Examples 1-7, wherein the second flexible portion is configured to deflect to form a curve extending proximally.

Example 9: The system of any example herein, in particular Example 8, wherein the plane is a first plane, and the curve is configured to extend in a second plane that is non-orthogonal and non-parallel with the first plane.

Example 10: The system of any example herein, in particular Example 9, wherein a distal tip of the second flexible portion includes an aperture for the implant to pass through to deploy from the delivery catheter.

Example 11: The system of any example herein, in particular Example 10, wherein the curve is configured to position the distal tip to extend in a plane that is parallel and offset with a plane that the first flexible portion extends in.

Example 12: The system of any example herein, in particular Examples 1-11, wherein the first linear spine and the second linear spine are embedded in a body of the elongate shaft.

Example 13: The system of any example herein, in particular Examples 1-12, wherein the first tether comprises a pull tether configured to be retracted proximally to deflect the first flexible portion and the second tether comprises a pull tether configured to be retracted proximally to deflect the second flexible portion.

Example 14: The system of any example herein, in particular Examples 1-13, further comprising the implant, and wherein the implant comprises a docking coil.

Example 15: The system of any example herein, in particular Examples 1-14, further comprising a steerable guide sheath including a lumen for the elongate shaft to pass through, the steerable guide sheath configured to deflect a portion of the elongate shaft when the elongate shaft is positioned within the lumen of the steerable guide sheath.

Example 16: A system for delivering an implant to a portion of a patient's body, the system comprising: a delivery catheter including: an elongate shaft having an interior lumen for the implant to pass through and a distal end portion including a flexible portion with a tether and a linear spine that is positioned at an obtuse angle circumferentially from the tether, and the flexible portion configured to deflect to form a curve upon a longitudinal force being applied to the tether.

Example 17: The system of any example herein, in particular Example 16, wherein the curve is configured to extend proximally.

Example 18: The system of any example herein, in particular Example 16 or Example 17, wherein a distal tip of the flexible portion includes an aperture for the implant to pass through to deploy from the delivery catheter.

Example 19: The system of any example herein, in particular Examples 16-18, wherein the flexible portion is a second flexible portion, and the tether is a second tether, and the linear spine is a second linear spine, and the elongate shaft further comprises: a first flexible portion positioned proximal of the second flexible portion and including a first tether and a first linear spine that is positioned opposed circumferentially to the first tether, and the first flexible portion is configured to deflect in a plane upon a longitudinal force being applied to the first tether.

Example 20: The system of any example herein, in particular Example 19, wherein the plane is a first plane, and the curve is configured to extend in a second plane that is non-orthogonal and non-parallel with the first plane.

Example 21: The system of any example herein, in particular Example 19 or Example 20, wherein the second flexible portion is configured to deflect in a direction that is obtuse relative to a direction of deflection of the first flexible portion.

Example 22: The system of any example herein, in particular Examples 19-21, wherein the first tether comprises a pull tether configured to be retracted proximally to deflect the first flexible portion and the second tether comprises a pull tether configured to be retracted proximally to deflect the second flexible portion.

Example 23: The system of any example herein, in particular Examples 19-22, wherein the first linear spine and the second linear spine are embedded in a body of the elongate shaft.

Example 24: The system of any example herein, in particular Examples 16-23, further comprising the implant, and wherein the implant comprises a docking coil.

Example 25: The system of any example herein, in particular Examples 16-24, further comprising a steerable guide sheath including a lumen for the elongate shaft to pass through, the steerable guide sheath configured to deflect a portion of the elongate shaft when the elongate shaft is positioned within the lumen of the steerable guide sheath.

Example 26: A system for delivering an implant to a portion of a patient's body. The system may include a delivery catheter including: an elongate shaft having an interior lumen for the implant to pass through and a distal end portion including a first flexible portion and a second flexible portion that is positioned distal of the first flexible portion, the first flexible portion including a first tether and a first linear spine that is positioned opposed circumferentially to the first tether, and the first flexible portion is configured to deflect in a first plane upon a longitudinal force being applied to the first tether, and the second flexible portion including a second tether positioned orthogonal relative to the first tether and a second linear spine positioned opposed circumferentially to the second tether, and a third tether positioned opposed circumferentially relative to the first tether, and the second flexible portion is configured to deflect in a second plane that is orthogonal to the first plane upon a longitudinal force being applied to the second tether and the second flexible portion is configured to deflect in the first plane upon a longitudinal force being applied to the third tether.

Example 27: The system of any example herein, in particular Example 26, wherein the second flexible portion is configured to deflect in a direction that is obtuse relative to a direction of deflection of the first flexible portion upon a longitudinal force being applied to both the second tether and the third tether.

Example 28: The system of any example herein, in particular Example 26 or Example 27, wherein the second flexible portion is configured to deflect to form a curve extending proximally.

Example 29: The system of any example herein, in particular Example 28, wherein the curve is configured to extend in a third plane that is non-orthogonal and non-parallel with the first plane upon a longitudinal force being applied to both the second tether and the third tether.

Example 30: The system of any example herein, in particular Examples 26-29, wherein a distal tip of the second flexible portion includes an aperture for the implant to pass through to deploy from the delivery catheter.

Example 31: The system of any example herein, in particular Examples 26-30, wherein the second flexible portion includes a third linear spine positioned opposed circumferentially to the third tether.

Example 32: The system of any example herein, in particular Examples 26-31, wherein the first linear spine and the second linear spine are embedded in a body of the elongate shaft.

Example 33: The system of any example herein, in particular Examples 26-32, wherein the first tether comprises a pull tether configured to be retracted proximally to deflect the first flexible portion and the second tether comprises a pull tether configured to be retracted proximally to deflect the second flexible portion and the third tether comprises a pull tether configured to be retracted proximally to deflect the second flexible portion.

Example 34: The system of any example herein, in particular Examples 26-33, further comprising the implant, and wherein the implant comprises a docking coil.

Example 35: The system of any example herein, in particular Examples 26-34, further comprising a steerable guide sheath including a lumen for the elongate shaft to pass through, the steerable guide sheath configured to deflect a portion of the elongate shaft when the elongate shaft is positioned within the lumen of the steerable guide sheath.

Example 36: A system. The system may include a docking coil configured to dock with an implant within a portion of a patient's body; and a docking coil sleeve having an interior lumen configured for the docking coil to slide within and including a tether extending along at least a portion of the docking coil sleeve and configured to deflect the docking coil sleeve.

Example 37: The system of any example herein, in particular Example 36, wherein the docking coil sleeve includes a lubricous outer surface facing opposite the interior lumen.

Example 38: The system of any example herein, in particular Example 36 or Example 37, wherein the docking coil sleeve includes a distal tip having an aperture for the docking coil to pass through to deploy from the docking coil sleeve.

Example 39: The system of any example herein, in particular Example 38, wherein the tether is configured to deflect the distal tip.

Example 40: The system of any example herein, in particular Example 39, wherein the docking coil sleeve is configured to form a coil, and the tether is configured to deflect the distal tip radially inward when the docking coil sleeve forms a coil.

Example 41: The system of any example herein, in particular Examples 36-40, wherein the docking coil sleeve includes a braid positioned at a distal end portion of the docking coil sleeve.

Example 42: The system of any example herein, in particular Example 41, wherein the braid has a flexibility that increases in a direction towards a distal tip of the docking coil sleeve.

Example 43: The system of any example herein, in particular Examples 36-42, wherein the docking coil sleeve includes a spine extending along at least a portion of the docking coil sleeve.

Example 44: The system of any example herein, in particular Example 43, wherein the spine is positioned opposed circumferentially to the tether.

Example 45: The system of any example herein, in particular Examples 36-44, wherein the tether comprises a pull tether configured to be retracted proximally to deflect the docking coil sleeve.

Example 46: A system. The system may include a docking coil configured to dock with an implant within a portion of a patient's body and including a leading portion extending to a leading tip and having an orientation; and a docking coil sleeve having an interior lumen configured for the docking coil to slide within and including a leading portion extending to a leading tip and having an orientation that is different than the orientation of the leading portion of the docking coil, the leading tip of the docking coil sleeve configured to slide relative to the leading tip of the docking coil to deflect the leading tip of the docking coil or the leading tip of the docking coil sleeve radially inward or outward.

Example 47: The system of any example herein, in particular Example 46, wherein the orientation of the leading portion of the docking coil is configured to form a diameter that is less than a diameter of the leading portion of the docking coil sleeve, and sliding the leading tip of the docking coil distally relative to the leading tip of the docking coil sleeve deflects the leading tip of the docking coil sleeve radially inward.

Example 48: The system of any example herein, in particular Example 46 or Example 47, wherein sliding the leading tip of the docking coil proximally relative to the leading tip of the docking coil sleeve deflects the leading tip of the docking coil sleeve radially outward.

Example 49: The system of any example herein, in particular Examples 46-48, wherein the leading portion of the docking coil has a preset radius of curvature.

Example 50: The system of any example herein, in particular Example 49, wherein the leading portion of the docking coil sleeve has a preset radius of curvature that is larger than the preset radius of curvature of the leading portion of the docking coil.

Example 51: The system of any example herein, in particular Example 46, wherein the orientation of the leading portion of the docking coil is configured to form a diameter that is greater than a diameter of the leading portion of the docking coil sleeve, and sliding the leading tip of the docking coil sleeve distally relative to the leading tip of the docking coil deflects the leading tip of the docking coil radially inward.

Example 52: The system of any example herein, in particular Example 51, wherein the docking coil includes one or more cuts on an inner curve portion of the docking coil, the one or more cuts configured to allow the leading tip of the docking coil to deflect.

Example 53: The system of any example herein, in particular Example 51 or Example 52, wherein the leading portion of the docking coil sleeve has a preset radius of curvature that is smaller than a preset radius of curvature of the leading portion of the docking coil.

Example 54: The system of any example herein, in particular Examples 46-53, further comprising a braid positioned at the leading portion of the docking coil sleeve.

Example 55: The system of any example herein, in particular Examples 46-54, further comprising a spine extending along the leading portion of the docking coil sleeve.

Example 56: A method comprising: advancing a delivery catheter to a position within a patient's body, the delivery catheter including: an elongate shaft having an interior lumen for an implant to pass through and a distal end portion including a first flexible portion and a second flexible portion that is positioned distal of the first flexible portion, the first flexible portion including a first tether and a first linear spine that is positioned opposed circumferentially to the first tether, and the first flexible portion is configured to deflect in a plane upon a longitudinal force being applied to the first tether, and the second flexible portion including a second tether and a second linear spine that is positioned non-orthogonal and non-parallel relative to the first linear spine, and the second flexible portion is configured to deflect in a direction that is non-orthogonal and non-parallel with the plane upon a longitudinal force being applied to the second tether. The method may include deploying the implant from the interior lumen to an implantation site within the patient's body.

Example 57: The method of any example herein, in particular Example 56, further comprising deflecting the second flexible portion to form a curve extending proximally.

Example 58: The method of any example herein, in particular Example 57, wherein the plane is a first plane, and the curve extends in a second plane that is non-orthogonal and non-parallel with the first plane.

Example 59: The method of any example herein, in particular Example 57 or Example 58, wherein the curve forms a height between the first flexible portion and a distal tip of the second flexible portion.

Example 60: The method of any example herein, in particular Example 59, wherein the first flexible portion is positioned in an atrium of the patient's heart and the height is in a ventricular direction.

Example 61: The method of any example herein, in particular Example 59 or Example 60, wherein the curve positions the distal tip to extend in a plane that is parallel and offset with a plane that the first flexible portion extends in.

Example 62: The method of any example herein, in particular Examples 56-61, further comprising retracting the second tether to deflect the second flexible portion.

Example 63: The method of any example herein, in particular Examples 56-62, further comprising retracting the first tether to deflect the first flexible portion.

Example 64: The method of any example herein, in particular Examples 56-63, wherein the elongate shaft is positioned within an atrium of the patient's heart, and the method further comprises deflecting the second flexible portion to a commissure of the patient's mitral valve.

Example 65: The method of any example herein, in particular Examples 56-64, wherein the implant comprises a docking coil, and the method further comprises deploying the docking coil around leaflets of the patient's mitral valve.

Example 66: A method comprising: advancing a delivery catheter to a position within a patient's body. The delivery catheter may include an elongate shaft having an interior lumen for an implant to pass through and a distal end portion including a first flexible portion and a second flexible portion that is positioned distal of the first flexible portion, the first flexible portion including a first tether and a first linear spine that is positioned opposed circumferentially to the first tether, and the first flexible portion is configured to deflect in a plane upon a longitudinal force being applied to the first tether, and the second flexible portion including a second tether and a second linear spine that is positioned non-orthogonal and non-parallel relative to the first linear spine, and the second flexible portion is configured to deflect in a direction that is non-orthogonal and non-parallel with the plane upon a longitudinal force being applied to the second tether. The method may include deploying the implant from the interior lumen to an implantation site within the patient's body.

Example 67: The method of any example herein, in particular Example 66, further comprising deflecting the second flexible portion to form a curve extending proximally.

Example 68: The method of any example herein, in particular Example 67, wherein the curve extends in a second plane that is non-orthogonal and non-parallel with the first plane.

Example 69: The method of any example herein, in particular Example 67 or Example 68, wherein the curve forms a height between the first flexible portion and a distal tip of the second flexible portion.

Example 70: The method of any example herein, in particular Example 69, wherein the first flexible portion is positioned in an atrium of the patient's heart and the height is in a ventricular direction.

Example 71: The method of any example herein, in particular Example 69 or Example 70, wherein the curve positions the distal tip to extend in a plane that is parallel and offset with a plane that the first flexible portion extends in.

Example 72: The method of any example herein, in particular Examples 66-71, further comprising retracting both the second tether and the third tether to deflect the second flexible portion.

Example 73: The method of any example herein, in particular Examples 66-72, further comprising retracting the first tether to deflect the first flexible portion.

Example 74: The method of any example herein, in particular Examples 66-73, wherein the elongate shaft is positioned within an atrium of the patient's heart, and the method further comprises deflecting the second flexible portion to a commissure of the patient's mitral valve.

Example 75: The method of any example herein, in particular Examples 66-74, wherein the implant comprises a docking coil, and the method further comprises deploying the docking coil around leaflets of the patient's mitral valve.

Example 76: A method comprising: deploying a docking coil from a docking coil sleeve to an implantation site within a patient's body, the docking coil being configured to dock with an implant within the patient's body, and the docking coil sleeve having an interior lumen configured for the docking coil to slide within and including a tether extending along at least a portion of the docking coil sleeve and configured to deflect the docking coil sleeve.

Example 77: The method of any example herein, in particular Example 76, further comprising deflecting the docking coil sleeve with the tether.

Example 78: The method of any example herein, in particular Example 76 or Example 77, further comprising retracting the tether to deflect the docking coil sleeve.

Example 79: The method of any example herein, in particular Examples 76-78, wherein a distal tip of the docking coil sleeve overhangs a distal tip of the docking coil.

Example 80: The method of any example herein, in particular Examples 76-79, wherein the docking coil sleeve includes a braid positioned at a distal end portion of the docking coil sleeve.

Example 81: The method of any example herein, in particular Example 80, wherein the braid has a flexibility that increases in a direction towards a distal tip of the docking coil sleeve.

Example 82: The method of any example herein, in particular Examples 76-81, wherein the docking coil sleeve includes a spine extending along at least a portion of the docking coil sleeve and positioned opposed circumferentially to the tether.

Example 83: The method of any example herein, in particular Examples 76-82, wherein the implantation site is the patient's mitral valve.

Example 84: The method of any example herein, in particular Example 83, further comprising forming the docking coil sleeve into a coil around leaflets of the patient's mitral valve and deflecting the docking coil sleeve radially inward or outward utilizing the tether.

Example 85: The method of any example herein, in particular Example 83 or Example 84, further comprising extending the docking coil sleeve and the docking coil around leaflets of the patient's mitral valve, and retracting the docking coil relative to the docking coil sleeve to deploy the docking coil to the patient's mitral valve.

Example 86: A method comprising: deploying a docking coil from a docking coil sleeve to an implantation site within a patient's body, the docking coil configured to dock with an implant within a portion of a patient's body and including a leading portion extending to a leading tip and having an orientation, and the docking coil sleeve having an interior lumen configured for the docking coil to slide within and including a leading portion extending to a leading tip and having an orientation that is different than the orientation of the leading portion of the docking coil, the leading tip of the docking coil sleeve configured to slide relative to the leading tip of the docking coil to deflect the leading tip of the docking coil or the leading tip of the docking coil sleeve radially inward or outward.

Example 87: The method of any example herein, in particular Example 86, further comprising sliding the leading tip of the docking coil distally relative to the leading tip of the docking coil sleeve to deflect the leading tip of the docking coil sleeve radially inward.

Example 88: The method of any example herein, in particular Example 86 or Example 87, further comprising sliding the leading tip of the docking coil proximally relative to the docking coil sleeve to deflect the leading tip of the docking coil sleeve radially outward.

Example 89: The method of any example herein, in particular Examples 86-88, wherein the orientation of the leading portion of the docking coil is configured to form a diameter that is less than a diameter of the leading portion of the docking coil sleeve.

Example 90: The method of any example herein, in particular Example 86, wherein the orientation of the leading portion of the docking coil is configured to form a diameter that is greater than a diameter of the leading portion of the docking coil sleeve, and sliding the leading tip of the docking coil sleeve distally relative to the leading tip of the docking coil deflects the leading tip of the docking coil radially inward.

Example 91: The method of any example herein, in particular Example 90, wherein the docking coil includes one or more cuts on an inner curve portion of the docking coil, the one or more cuts configured to allow the leading tip of the docking coil to deflect.

Example 92: The method of any example herein, in particular Examples 86-91, wherein the docking coil sleeve includes a spine extending along the leading portion of the docking coil sleeve.

Example 93: The method of any example herein, in particular Examples 86-92, wherein the docking coil sleeve includes a braid positioned at the leading portion of the docking coil sleeve.

Example 94: The method of any example herein, in particular Examples 86-93, wherein the implantation site is the patient's mitral valve.

Example 95: The method of any example herein, in particular Examples 86-94, further comprising extending the docking coil sleeve and the docking coil around leaflets of the patient's mitral valve, and retracting the docking coil sleeve relative to the docking coil to deploy the docking coil to the patient's mitral valve.

Any of the features of any of the examples, including but not limited to any of the first through ninety-fifth examples referred to above, is applicable to all other aspects and embodiments identified herein, including but not limited to any embodiments of any of the first through ninety-fifth examples referred to above. Moreover, any of the features of an embodiment of the various examples, including but not limited to any embodiments of any of the first through ninety-fifth aspects referred to above, is independently combinable, partly or wholly with other examples described herein in anyway, e.g., one, two, or three or more examples may be combinable in whole or in part. Further, any of the features of the various examples, including but not limited to any embodiments of any of the first through ninety-fifth examples referred to above, may be made optional to other examples. Any example of a method can be performed by a system or apparatus of another example, and any aspect or embodiment of a system or apparatus can be configured to perform a method of another aspect or embodiment, including but not limited to any embodiments of any of the first through ninety-fifth examples referred to above.

Although the operations of some of the disclosed examples are described in a particular, sequential order for convenient presentation, it should be understood that this manner of description encompasses rearrangement, unless a particular ordering is required by specific language. For example, operations described sequentially can in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms can vary depending on the particular implementation and are readily discernible by one of ordinary skill in the art. Steps of various methods herein can be combined.

In view of the many possible examples to which the principles of the disclosure can be applied, it should be recognized that the illustrated examples are only preferred examples of the disclosure and should not be taken as limiting the scope of the disclosure. Rather, the scope of the disclosure is defined by the following claims.

What is claimed is:

1. A system for delivering an implant to a portion of a patient's body, the system comprising:
a delivery catheter including:
an elongate shaft having an interior lumen for the implant to pass through and a distal end portion including a first flexible portion and a second flexible portion that is positioned distal of the first flexible portion,
the first flexible portion including a first tether and a first linear spine that is positioned opposed circumferentially to the first tether, and the first flexible portion is configured to deflect in a plane upon a longitudinal force being applied to the first tether, and
the second flexible portion including a second tether and a second linear spine that is positioned non-orthogonal and non-parallel relative to the first linear spine, and the second flexible portion is configured to deflect in a direction that is non-orthogonal and non-parallel with the plane upon a longitudinal force being applied to the second tether.

2. The system of claim 1, wherein the second tether is positioned opposed circumferentially to the second linear spine.

3. The system of claim 1, wherein the second tether is positioned at an obtuse angle relative to the first tether.

4. The system of claim 1, wherein the second tether is positioned orthogonal relative to the first tether.

5. The system of claim 1, wherein the second linear spine is positioned at an obtuse angle relative to the first linear spine.

6. The system of claim 1, wherein the second linear spine is positioned at an acute angle relative to the first tether.

7. The system of claim 1, wherein the direction that the second flexible portion is configured to deflect in is obtuse relative to a direction of deflection of the first flexible portion.

8. The system of claim 1, wherein the second flexible portion is configured to deflect to form a curve extending proximally.

9. The system of claim 8, wherein the plane is a first plane, and the curve is configured to extend in a second plane that is non-orthogonal and non-parallel with the first plane.

10. The system of claim 9, wherein a distal tip of the second flexible portion includes an aperture for the implant to pass through to deploy from the delivery catheter.

11. The system of claim 10, wherein the curve is configured to position the distal tip to extend in a plane that is parallel and offset with a plane that the first flexible portion extends in.

12. The system of claim 1, wherein the first linear spine and the second linear spine are embedded in a body of the elongate shaft.

13. The system of claim 1, wherein the first tether comprises a pull tether configured to be retracted proximally to deflect the first flexible portion and the second tether comprises a pull tether configured to be retracted proximally to deflect the second flexible portion.

14. The system of claim 1, further comprising the implant, and wherein the implant comprises a docking coil.

15. The system of claim 1, further comprising a steerable guide sheath including a lumen for the elongate shaft to pass through, the steerable guide sheath configured to deflect a portion of the elongate shaft when the elongate shaft is positioned within the lumen of the steerable guide sheath.

16. A system comprising:
a docking coil configured to dock with an implant within a portion of a patient's body; and
a docking coil sleeve having an interior lumen configured for the docking coil to slide within and including a tether extending along at least a portion of the docking coil sleeve and configured to deflect the docking coil sleeve.

17. The system of claim 16, wherein the docking coil sleeve includes a lubricous outer surface facing opposite the interior lumen.

18. The system of claim 16, wherein the docking coil sleeve includes a distal tip having an aperture for the docking coil to pass through to deploy from the docking coil sleeve.

19. The system of claim 18, wherein the tether is configured to deflect the distal tip.

20. The system of claim 19, wherein the docking coil sleeve is configured to form a coil, and the tether is configured to deflect the distal tip radially inward when the docking coil sleeve forms a coil.

21. A system comprising:
a docking coil configured to dock with an implant within a portion of a patient's body and including a leading portion extending to a leading tip and having an orientation; and
a docking coil sleeve having an interior lumen configured for the docking coil to slide within and including a leading portion extending to a leading tip and having an orientation that is different than the orientation of the leading portion of the docking coil, the leading tip of the docking coil sleeve configured to slide relative to the leading tip of the docking coil to deflect the leading tip of the docking coil or the leading tip of the docking coil sleeve radially inward or outward.

22. The system of claim 21, wherein the orientation of the leading portion of the docking coil is configured to form a diameter that is less than a diameter of the leading portion of the docking coil sleeve, and sliding the leading tip of the docking coil distally relative to the leading tip of the docking coil sleeve deflects the leading tip of the docking coil sleeve radially inward.

23. The system of claim 21, wherein sliding the leading tip of the docking coil proximally relative to the leading tip of the docking coil sleeve deflects the leading tip of the docking coil sleeve radially outward.

24. The system of claim 21, wherein the leading portion of the docking coil has a preset radius of curvature.

25. The system of claim 24, wherein the leading portion of the docking coil sleeve has a preset radius of curvature that is larger than the preset radius of curvature of the leading portion of the docking coil.
